(19) United States
(12) Patent Application Publication
(10) Pub. No.: US 2004/0116665 A1
(43) Pub. Date: Jun. 17, 2004

(54) Vaccine composition

(76) Inventors: Francois-Xavier Jacques Berthet, Rixensart (BE); Philippe Denoel, Rixensart (BE); Cecile Anne Neyt, Rixensart (BE); Jan Poolman, Rixensart (BE); Joelle Thonnard, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/467,421

(22) PCT Filed: Feb. 8, 2002
(86) PCT No.: PCT/EP02/01361

(30) Foreign Application Priority Data
Feb. 8, 2001 (GB) 0103171.5

(51) Int. Cl. C12Q 1/68; C07K 1/00; C07K 14/00; C07K 17/00

(52) U.S. Cl. 530/350

(57) ABSTRACT

The present invention relates to the field of novel, engineered Gram-negative bacterial strains that have improved outer-membrane vesicle shedding properties, and vaccine compositions comprising these bacteria or vesicles. The present invention provides a hyperbledding Gram-negative bacterium which has been genetically modified by either or both processes selected from a group of consisting of: down-regulation of expression of one or more tol genes; and mutation of one or more gene(s) encoding a protein comprising a peptidoglycan-associated site to attenuate the peptidoglycan-binding activity of the protein(s).

### FIELD OF THE INVENTION

The present invention relates to the field of Gram-negative bacterial vaccine compositions, their manufacture, and the use of such compositions in medicine. More particularly it relates to the field of novel, engineered Gram-negative bacterial strains that have improved outer-membrane vesicle shedding properties, and vaccine compositions comprising these vesicles.

### BACKGROUND OF THE INVENTION

Gram-negative bacteria are separated from the external medium by two successive layers of membrane structures. These structures, referred to as the cytoplasmic membrane and the outer membrane (OM), differ both structurally and functionally. The outer membrane plays an important role in the interaction of pathogenic bacteria with their respective hosts. Consequently, the surface exposed bacterial molecules represent important targets for the host immune response, making outer-membrane components attractive candidates in providing vaccine, diagnostic and therapeutics reagents.

Whole cell bacterial vaccines (killed or attenuated) have the advantage of supplying multiple antigens in their natural micro-environment. Drawbacks around this approach are the side effects induced by bacterial components such as endotoxin and peptidoglycan fragments. On the other hand, a cellular subunit vaccines containing purified components from the outer membrane may supply only limited protection and may not present the antigens properly to the immune system of the host.

Proteins, phospholipids and lipopolysaccharides are the three major constituents found in the outer-membrane of all Gram-negative bacteria. These molecules are distributed asymmetrically: membrane phospholipids (mostly in the inner leaflet), lipooligosaccharides (exclusively in the outer leaflet) and proteins (inner and outer leaflet lipoproteins, integral or polytopic membrane proteins). For many bacterial pathogens which impact on human health, lipopolysaccharide and outer-membrane proteins have been shown to be immunogenic and amenable to confer protection against the corresponding disease by way of immunization.

The OM of Gram-negative bacteria is dynamic and, depending on the environmental conditions, can undergo drastic morphological transformations. Among these manifestations, the formation of outer-membrane vesicles or “blebs” has been studied and documented in many Gram-negative bacteria (Zhou, L et al. 1998. FEMS Microbiol. Lett. 163: 223-228). Among these, a non-exhaustive list of bacterial pathogens reported to produce blebs include: Bordetella pertussis, Borrelia burgdorferi, Brucella melitensis, Brucella ovis, Chlamydia psittaci, Chlamydia trachomatis, Esherichia coli, Haemophilus influenzae, Legionella pneumophila, Neisseria gonorrhoeae, Neisseria meningitidis, Pseudomonas aeruginosa and Yersinia enterocolitica. Although the biochemical mechanism responsible for the production of OM blebs is not fully understood, these outer membrane vesicles have been extensively studied as they represent a powerful methodology in order to isolate outer-membrane protein preparations in their native conformation. In that context, the use of outer-membrane preparations is of particular interest to develop vaccines against Neisseria, Moraxella catarrhalis, Haemophilus influenzae, Pseudomonas aeruginosa and Chlamydia. Moreover, outer membrane blebs combine multiple proteinaceaous and non-proteinaceous antigens that are likely to confer extended protection against intra-species variants.

Examples of bacterial species from which bleb vaccines can be made are the following. Neisseria menineitidis:

Neisseria meningitidis (meningococcus) is a Gram-negative bacterium frequently isolated from the human upper respiratory tract. It occasionally causes invasive bacterial diseases such as bacteremia and meningitis. The incidence of meningococcal disease shows geographical seasonal and annual differences (Schwartz, B., Moore, P. S., Broome, C. V.; Clin. Microbiol. Rev. 2 (Supplement), S18-S24, 1989). Most disease in temperate countries is due to strains of serogroup B and varies in incidence from 1-10/100,000/year total population sometimes reaching higher values (Kaczmarski, E. B. (1997), Commun. Dis. Rep. Rev. 7: R55-9, 1995; Scholten, R. J. P. M., Bijlmer, H. A., Poolman, J. T. et al. Clin. Infect. Dis. 16: 237-246, 1993; Cruz, C., Pavez, G., Aguilar, E., et al. Epidemiol. Infect. 105: 119-126, 1990). Age-specific incidences in the two high risk-groups, infants and teenagers, reach higher levels.

Epidemics dominated by serogroup A meningococci occur, mostly in central Africa, sometimes reaching levels up to 1000/100,000/year (Schwartz, B., Moore, P. S., Broome, C. V. Clin. Microbiol. Rev. 2 (Supplement), S18-S24, 1989). Nearly all cases of meningococcal disease as a whole are caused by serogroup A, B, C, W-135 and Y meningococci. A tetravalent A, C, W-135, Y capsular polysaccharide vaccine is available (Armand, J., Anninjon, F., Mynard, M. C., Lafaix, C., J. Biol. Stand. 10: 335-339, 1982).

The polysaccharide vaccines are currently being improved by way of chemically conjugating them to carrier proteins (Lieberman, J. M., Chiu, S. S., Wong, V. K., et al. JAMA 275: 1499-1503, 1996). A serogroup B vaccine is not available, since the B capsular polysaccharide is non-immunogenic, most likely because it shares structural similarity to host components (Wyle, F. A., Artenstein, M. S., Brandt, M. L. et al. J. Infect. Dis. 126: 514-522, 1972; Finne, J. M., Leinonen, M., Mäkelä, P. M. Lancet ii.: 355-357, 1983).

For many years efforts have been focused on developing meningococcal outer membrane based vaccines (de Moraes, J. C., Perkins, B., Camargo, M. C. et al. Lancet 340: 1074-1078, 1992; Bjune, G., Hoiby, E. A. Gronnesby, J. K. et al. 338: 1093-1096, 1991). Such vaccines have demonstrated efficacies from 57%-85% in older children (>4 years) and adolescents. Most of these efficacy trials were performed with OMV (outer membrane vesicles, derived by LPS depletion from blebs) vaccines derived from wild-type N. meningitidis B strains.

N. meningitidis serogroup B (menB) excretes outer membrane blebs in quantities that allow their preparation on an industrial scale. Such multicomponent outer-membrane protein vaccines from naturally-occurring menB strains have been found to be efficacious in protecting teenagers from menB disease and have become registered in Latin America. An alternative method of preparing outer-membrane vesicles is via the process of detergent extraction of the bacterial cells (EP 11243).

Many bacterial outer membrane components are present in these vaccines, such as PorA, PorB, Rmp, Opc, Opa, FrpB and the contribution of these components to the observed protection still needs further definition. Other bacterial outer membrane components have been defined (using animal or human antibodies) as potentially being relevant to the induction of protective immunity, such as TbpB, NspA (Martin, D., Cadieux, N., Hamel, J., Brodeux, B. R., J. Exp. Med. 185: 1173-1183, 1997; Lissolo, L., Maître-Wilmotte, C., Dumas, p. et al., Inf. Immun. 63: 884-890, 1995). The mechanism of protective immunity will involve antibody mediated bactericidal activity and opsonophagocytosis.

Moraxella catarrhalis

Moraxella catarrhalis (also named Branhamella catarrhalis) is a Gram-negative bacterium frequently isolated from the human upper respiratory tract. It is responsible for several pathologies, the main ones being otitis media in infants and children, and pneumonia in the elderly. It is also responsible for sinusitis, nosocomial infections and, less frequently, for invasive diseases.

Bactericidal antibodies have been identified in most adults tested (Chapman, A J et al. (1985) J. Infect. Dis. 151:878). Strains of M. catarrhalis present variations in their capacity to resist serum bactericidal activity: in general, isolates from diseased individuals are more resistant than those who are simply colonized (Hol, C et al. (1993) Lancet 341:1281, Jordan, K L et al. (1990) Am. J. Med. 88 (suppl. 5A):28S). Serum resistance could therfore be considered as a virulence factor of the bacteria An opsonizing activity has been observed in the sera of children recovering from otitis media The antigens targetted by these different immune responses in humans have not been identified, with the exception of OMP B1, a 84 kDa protein, the expression of which is regulated by iron, and that is recognized by the sera of patients with pneumonia (Sethi, S, et al. (1995) Infect. Immun. 63:1516), and of UspA1 and UspA2 (Chen D. et al.(1999), Infect. Inmun. 67:1310).

A few other membrane proteins present on the surface of M. catarrhalis have been characterized using biochemical methods for their potential implication in the induction of a protective immunity (for review, see Murphy, TF (1996) Microbiol. Rev. 60:267). In a mouse pneumonia model, the presence of antibodies raised against some of them (UspA, CopB) favors a faster clearance of the pulmonary infection. Another polypeptide (OMP CD) is highly conserved among M. catarrhalis stains, and presents homologies with a porin of Pseudomonas aeruginosa, which has been demonstrated to be efficacious against this bacterium in animal models.

M. catarrhalis produces outer membrane vesicles (Blebs). These Blebs have been isolated or extracted by using different methods. Among these methods, detergent extraction (Bartos L. C. and Murphy T. M. 1988. J. Infect. Dis. 158: 761-765; Murphy T. M. and Loeb M. R. 1989 Microbial Pathog. 6:159-174; Unhanand M., Maciver I., Ramilo O., Arencibia-Mireles O., Argyle J. C., McCracken G. H., Hansen E. J. 1992. J. Infect. Dis. 165: 644-650; Maciver I., Unhanand M., McCracken G. H. and Hansen E. J. 1993. J. Infect. Dis. 168: 469-472) or the production of ghosts (Lubitz W., et al. 1999. J. Biotechnol. 73: 261-273; Eko F. O., et. al. 1999. Vaccine 17: 1643-1649) are well known. The protective capacity of such Bleb preparations has been tested in a murine model for pulmonary clearance of M. catarrhalis. It has been shown that active immunization with Bleb vaccine or passive transfer of anti-Blebs antibody induces significant protection in this model (Maciver I., Unhanand M., McCracken G. H. Jr., Hansen, E. J. 1993. J. Infect. Dis. 168: 469-472).

Haemophilus influenzae

Haemophilus influenzae is a non-motile Gram-negative bacterium. Man is its only natural host. H. influenzae isolates are usually classified according to their polysaccharide capsule. Six different capsular types designated ‘a’ through ‘f’ have been identified. Isolates that fail to agglutinate with antisera raised against one of these six serotypes are classified as nontypeable, and do not express a capsule.

H. influenzae type b (Hib) is clearly different from the other types in that it is a major cause of bacterial meningitis and systemic diseases. Nontypeable strains of H. influenzae (NTHi) are only occasionally isolated from the blood of patients with systemic disease. NTHi is a common cause of pneumonia, exacerbation of chronic bronchitis, sinusitis and otitis media. NTHi strains demonstrate a large variability as identified by clonal analysis, whilst Hib strains as a whole are more homogeneous.

Various proteins of H. influenzae have been shown to be involved in pathogenesis or have been shown to confer protection upon vaccination in animal models.

Adherence of NTHi to human nasopharygeal epithelial cells has been reported (Read R C. et al. 1991. J. Infect. Dis. 163:549). Apart from fimbriae and pili (Brinton CC. et al. 1989. Pediatr. Infect. Dis. J. 8:S54; Kar S. et al. 1990. Infect. Immun. 58:903; Gildorf JR. et al. 1992. Infect. Immun. 60:374; St. Geme J W et al. 1991. Infect. Immun. 59:3366; St. Geme J W et al. 1993. Infect. Immun. 61: 2233), many adhesions have been identified in NTHi. Among them, two surface exposed high-molecular-weight proteins designated HMW1 and HMW2 have been shown to mediate adhesion of NTHi to epithelial cells (St. Geme T W. et al. 1993. Proc. Natl. Acad. Sci. USA 90:2875). Another family of high-molecular-weight proteins has been identified in NTHi strains that lack proteins belonging to HMW1/HMW2 family. The NTHi 115-kDa Hia protein (Barenkamp S J., St Geme S. W. 1996. Mol. Microbiol. In press) is highly similar to the Hsf adhesin expressed by H. influenzae type b strains (St. Geme J W. et al. 1996. J. Bact. 178:6281). Another protein, the Hap protein shows similarity to IgA1 serine proteases and has been shown to be involved in both adhesion and cell entry (St. Geme J W. et al. 1994. Mol. Microbiol. 14:217).

Five major outer membrane proteins (OMP) have been identified and numerically numbered. Original studies using H. influenzae type b strains showed that antibodies specific for P1 and P2 OMPs protected infant rats from subsequent challenge (Loeb M R. et al. 1987. Infect. Immun. 55:2612; Musson R S. Jr. et al. 1983. J. Clin. Invest. 72:677). P2 was found to be able to induce bactericidal and opsonic antibodies, which are directed against the variable regions present within surface exposed loop structures of this integral OMP (Haase E M. et al. 1994 Infect. Immun. 62:3712; Troelstra A. et al. 1994 Infect. Immun. 62:779). The lipoprotein P4 also may induce bactericidal antibodies (Green B A. et al. 1991. Infect. Immun. 59:3191).

OMP P6 is a conserved peptidoglycan associated lipoprotein making up 1-5% of the outer membrane (Nelson M B. et al. 1991. Infect. Immun. 59:2658). Later a lipoprotein of about the same molecular weight was recognized called PCP (P6 cross-reactive protein) (Deich R M. et al. 1990. Infect. Immun. 58:3388). A mixture of the conserved lipoproteins P4, P6 and PCP did not reveal protection as measured in a chinchilla otitis-media model (Green B A. et al. 1993. Infect. Immun. 61:1950). P6 alone appears to induce protection in the chinchilla model (Demaria T F. et al. 1996. Infect. Immun. 64:5187).

A fimbrin protein (Miyamoto N., Bakaletz, L O. 1996. Microb. Pathog. 21:343) has also been described with homology to OMP P5, which itself has sequence homology to the integral Escherichia coli OmpA (Miyamoto N., Bakaletz, L O. 1996. Microb. Pathog. 21:343; Munson R S. Jr. et al. 1993. Infect. Immun. 61:1017). NTHi seem to adhere to mucus by way of fimbriae.

In line with the observations made with gonococci and meningococci, NTHi expresses a dual human transferrin receptor composed of ThpA and TbpB when grown under iron limitation. Anti-TbpB antibody protected infant rats (Loosmore S M. et al. 1996. Mol. Microbiol. 19:575). Hemoglobin/haptoglobin receptor also have been described for NTH (Maciver I. et al. 1996. Infect. Immun. 64:3703). A receptor for Haem:Hemopexin has also been identified (Cope L D. et al. 1994. Mol. Microbiol. 13:868). A lactoferrin receptor is also present amongst NTHi, but is not yet characterized (Schryvers A B. et al. 1989. J. Med. Microbiol. 29:121). A protein similar to neisserial FrpB-protein has not been described amongst NTHi.

An 80 kDa OMP, the D15 surface antigen, provides protection against NTHi in a mouse challenge model. (Flack F S. et al. 1995. Gene 156:97). A 42 kDa outer membrane lipoprotein, LPD is conserved amongst Haemophilus influenzae and induces bactericidal antibodies (Akkoyunlu M. et al. 1996. Infect. Immun. 64:4586). A minor 98 kDa OMP (Kimura A. et al. 1985. Infect. Immun. 47:253), was found to be a protective antigen, this OMP may very well be one of the Fe-limitation inducible OMPs or high molecular weight adhesins that have been characterized thereafter. H. Influenzae produces IgA1-protease activity (Mulks M H., Shoberg R J. 1994. Meth. Enzymol. 235:543). IgA1-proteases of NTHi have a high degree of antigenic variability (Lomholt H., van Alphen L., Kilian, M. 1993. Infect. Immun. 61:4575).

Another OMP of NTHi, OMP26, a 26-kDa protein has been shown to enhance pulmonary clearance in a rat model (Kyd, J. M., Cripps, A. W. 1998. Infect. Immun. 66:2272). The NTHi HtrA protein has also been shown to be a protective antigen. Indeed, this protein protected Chinchilla against otitis media and protected infant rats against H. influenzae type b bacteremia (Loosmore S. M. et al. 1998. Infect. Immun. 66:899).

Outer membrane vesicles (or blebs) have been isolated from H. influenzae (Loeb M. R., Zachary A. L., Smith D. H. 1981. J. Bacteriol. 145:569-604; Stull T. L., Mack K., Haas J. E., Smit J., Smith A. L. 1985. Anal. Biochern. 150: 471480), as have the production of ghosts (Lubitz W., et al. 1999. J. Biotechnol. 73: 261-273; Eko F. O., et. al. 1999. Vaccine 17: 1643-1649). The vesicles have been associated with the induction of blood-brain barrier permeability (Wiwpelwey B., Hansen E. J., Scheld W. M. 1989 Infect. Immun. 57: 2559-2560), the induction of meningeal inflammation (Mustafa M. M., Ramilo O., Syrogiannopoulos GA., Olsen K. D., McCraken G. H. Jr., Hansen, E. J. 1989. J. Infect. Dis. 159: 917-922) and to DNA uptake (Concino M. F., Goodgal S. H. 1982 J. Bacteriol. 152: 441450). These vesicles are able to bind and be absorbed by the nasal mucosal epithelium (Harada T., Shimuzu T., Nishimoto K, Sakakura Y. 1989. Acta Otorhinolarygol. 246: 218-221) showing that adhesins and/or colonization factors could be present in Blebs. Immune response to proteins present in outer membrane vesicles has been observed in patients with various H. influenzae diseases (Sakakura Y., Harada T., Hamaguchi Y., Jin C. S. 1988. Acta Otorhinolarygol. Suppl. (Stockh.) 454: 222-226; Harada T., Sakakura Y., Miyoshi Y. 1986. Rhinology 24: 61-66).

Pseudomonas aeruginosa:

The genus Pseudomonas consists of Gram-negative, polarly flagellated, straight and slightly curved rods that grow aerobically and do not forms spores. Because of their limited metabolic requirements, Pseudomonas spp. are ubiquitous and are widely distributed in the soil, the air, sewage water and in plants. Numerous species of Pseudomonas such as P. aeruginosa, P. pseudomallei P. mallei, P. maltophilia and P. cepacia have also been shown to be pathogenic for humans. Among this list, P. aeruginosa is considered as an important human pathogen since it is associated with opportunistic infection of immuno-compromised host and is responsible for high morbidity in hospitalized patients. Nosocomial infection with P. aeruginosa afflicts primarily patients submitted for prolonged treatment and receiving immuno-suppressive agents, corticosteroids, antimetabolites antibiotics or radiation.

The Pseudomonas, and particularly P. aeruginosa, produces a variety of toxins (such as hemolysins, fibrinolysins, esterases, coagulases, phospholipases, endo- and exo-toxins) that contribute to the pathogenicity of these bacteria. Moreover, these organisms have high intrinsic resistance to antibiotics due to the presence of multiple drug efflux pumps. This latter characteristic often complicates the outcome of the disease.

Due to the uncontrolled use of antibacterial chemotherapeutics the frequency of nosocomial infection caused by P. aeruginosa has increased considerably over the last 30 years. In the US, for example, the economic burden of P. aeruginosa nosocomial infection is estimated to 4.5 billion US$ annually. Therefore, the development of a vaccine for active or passive immunization against P. aeruginosa is actively needed (for review see Stanislavsky et al. 1997. FEMS Microbiol. Lett. 21: 243-277).

Various cell-associated and secreted antigens of P. aeruginosa have been the subject of vaccine development. Among Pseudomonas antigens, the mucoid substance, which is an extracellular slime consisting predominantly of alginate, was found to be heterogenous in terms of size and immunogenicity. High molecular mass alginate components (30-300 kDa) appear to contain conserved epitopes while lower molecular mass alginate components (10-30 kDa) possess conserved epitopes in addition to unique epitopes. Among surface-associated proteins, PcrV, which is part of the type m secretion-translocation apparatus, has also been shown to be an interesting target for vaccination (Sawa et al. 1999. Nature Medicine 5:392-398).

Surface-exposed antigens including O-antigens (O-specific polysaccharide of LPS) or H-antigens (flagellar antigens) have been used for serotyping due to their highly immunogenic nature. Chemical structures of repeating units of O-specific polysaccharides have been elucidated and these data allowed the identification of 31 chemotypes of P. aeruginosa. Conserved epitopes among all serotypes of P. aeruginosa are located in the core oligosaccharide and the lipid A region of LPS and immunogens containing these epitopes induce cross-protective immunity in mice against different P. aeruginosa immunotypes. The outer core of LPS was implicated to be a ligand for binding of P. aeruginosa to airway and ocular epithelial cells of animals. However, heterogeneity exists in this outer core region among different serotypes. Epitopes in the inner core are highly conserved and have been demonstrated to be surface-accessible, and not masked by O-specific polysaccharide.

To examine the protective properties of OM proteins, a vaccine containing P. aeruginosa OM proteins of molecular masses ranging from 20 to 100 kDa has been used in pre-clinical and clinical trials. This vaccine was efficacious in animal models against P. aeruginosa challenge and induced high levels of specific antibodies in human volunteers. Plasma from human volunteers containing anti-P. aeruginosa antibodies provided passive protection and helped the recovery of 87% of patients with severe forms of P. aeruginosa infection. More recently, a hybrid protein containing parts of the outer membrane proteins OprF (amino acids 190-342) and OprI (amino acids 21-83) from Pseudomonas aeruginosa fused to the glutathione-S-transferase was shown to protect mice against a 975-fold 50% lethal dose of P. aeruginosa (Knapp et al. 1999. Vaccine. 17:1663-1669).

However, the purification of blebs is technically difficult; bleb production in most Gram-negative strains results in poor yields of product for the industrial production of vaccines, and often in a very heterogeneous product. The present invention solves this problem by providing specially modified “hyperblebbing” strains from which blebs may be more easily made in higher yield and may be more homogeneous in nature. Such blebs may also be more readily filter sterilised.

In addition, if the bacteria make more blebs naturally, there are considerable process advantages associated with bleb purification in that blebs can be made and harvested without the use of detergents such as deoxycholate (for extraction of greater quantities of blebs). This would mean that usual process steps to remove detergent such as chromatography purification and ultra centrifugation may be obviated.

### BRIEF DESCRIPTION OF THE DRAWINGS

FIG. 1: Multiple alignment of peptidoglycan-associated proteins. EC is E. coli, HI is Haemophilus influenzae, NG is Neisseria gonorrhoeae. ↑ indicates the position of the conserved F residue of OmpA homologues which should be conserved in C-terminal truncates. ______ indicates the conserved full extent of the peptidoglycan-associating site.

FIG. 2: Multiple alignment of peptidoglycan-associated proteins. EC is E. coli, MC is Moraxella catarrhalis, NG is Neisseria gonorrhoeae. ↑ indicates the position of the conserved F residue of OmpA homologues which should be conserved in C-terminal truncates. ______ indicates the conserved full extent of the peptidoglycan-associating site.

FIG. 3: Shows a hypothetical schematic structure of ompCD of M. catarrhalis. The location of the F residue of OmpA homologues which should be conserved in C-terminal truncates is shown, as is the peptidoglycan-associating site.

FIG. 4: Shows PCR screening of recombinant Neisseria resulting from a double crossing over at the rmp locus as described in Example 1.

FIG. 5: Schematic representation of the strategy used to construct the mutator plasmids for the deletion of tol genes in Moraxella catarrhalis and NTHI

FIG. 6: A: Schematic representation of the expected double recombinant tolQR Moraxella catarrhalis. B: PCR analysis of recombinant tolQR Moraxella catarrhalis clones using primers E, F, G and H

FIG. 7: Construction of the mutator plasmids used for the introduction of a stop codon into the ompCD sequence and P5 sequence of Moraxella catarrhalis and NTHI respectively.

### DESCRIPTION OF THE INVENTION

In a first aspect, the present invention provides a hyperblebbing Gram-negative bacterium which has been genetically modified by either or both processes selected from a group consisting of: down-regulation of expression of one or more tol genes; and mutation of one or more gene(s) encoding a protein comprising a peptidoglycan-associated site to attenuate the peptidoglycan-binding activity of the protein(s).

By ‘hyperblebbing’ it is meant that the bacterium naturally sheds 2 times or more (more preferably 3, 4, 5, or 10 times or more) the quantity of blebs of the unmodified bacterium.

By ‘down-regulation’ and ‘down-regulating’ it is meant that expression of the gene in question is reduced (by at least 2 fold, preferably 5 fold or more) or switched off completely. This can readily be done by methods such as deleting the gene from the genome, introducing a stop codon into the coding sequence of the gene, deleting the promoter sequence of the gene, or replacing the promoter sequence of the gene for a weaker promoter. Where the gene is in an operon (as many tol genes are) care must be taken to ensure that the down-regulation of the target gene does not affect expression of the other genes in the operon that are not intended to be down regulated.

Specific tol genes may be identified in various Gram-negative bacteria by homology (preferably more than 20, 30, 40, 50, 60, 70, 80, 90% identity or more) to the tol genes described herein (for instance toA, B, Q or R), or those of E. coli. Preferably 1, 2, 3, 4 or 5 tol genes are down-regulated in the bacterium of the invention. Most preferably pairs of tol genes: tolQ and tolR, or tolR and tolA are down-regulated (preferably by deletion or introduction of a disruptive stop codon) in a bacterium.

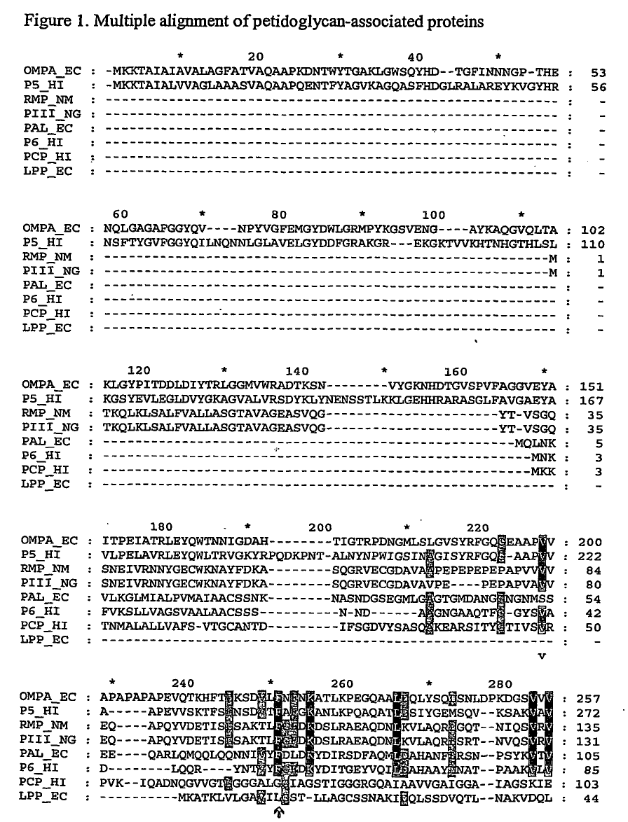

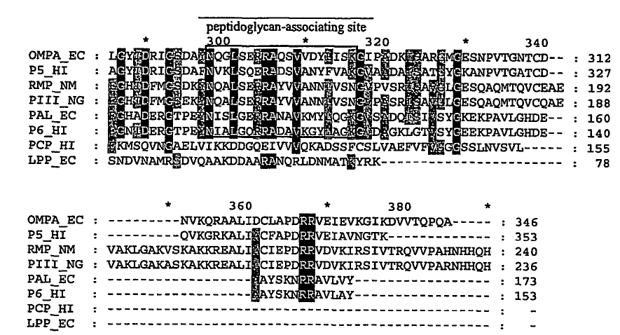

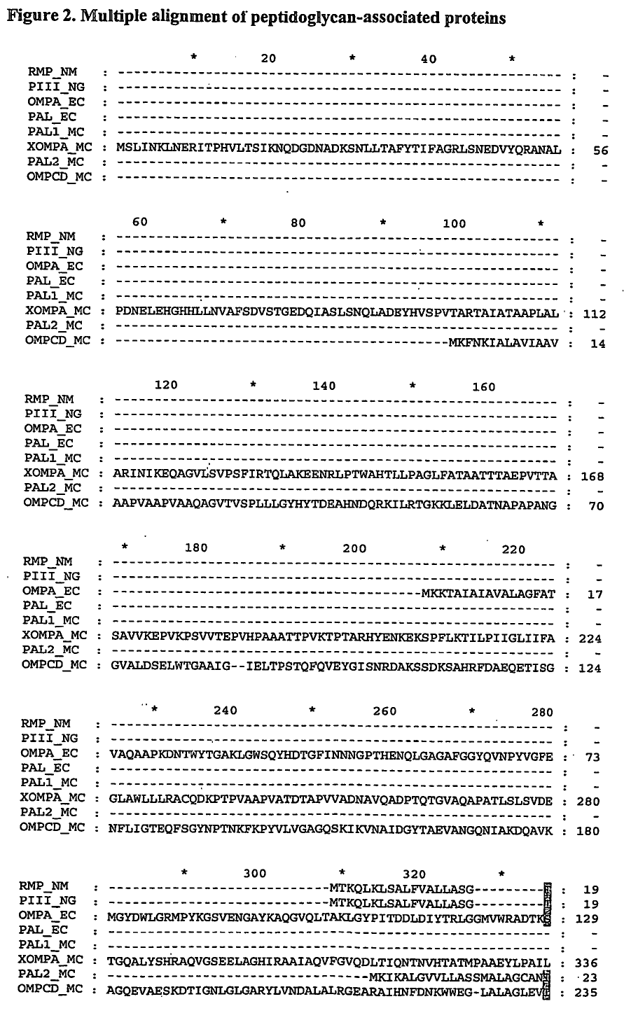

By ‘mutation’ of one or more gene(s) encoding a protein comprising a peptidoglycan-associated site to attenuate the peptidoglycan-binding activity of the protein(s) it is meant that such genes are either ‘down-regulated’ as described above. Alternatively, because such genes may encode protective antigens, a stop codon may be introduced within or 5′ to the part of the gene encoding the peptidoglycan-associating site (a peptide of approximately 16-22 amino acids which is conserved and identifiable amongst Gram-negative bacterial strains, as shown in FIGS. 1 and 2, or amino acid sequences 40, 50, 60, 70, 80, 90% or more identical to said sequences).

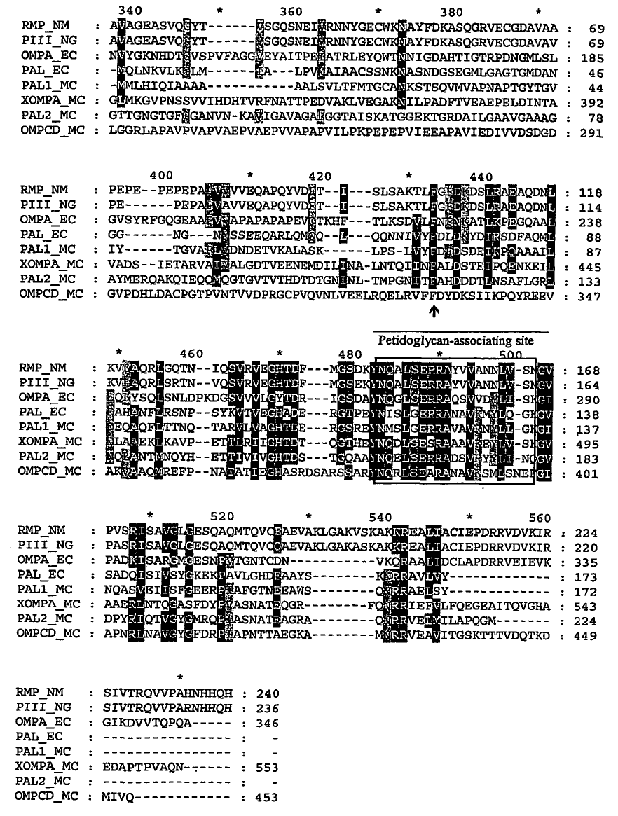

Frequently, such genes are integral membrane proteins, and therefore it is preferable for the stop codon to be 3′ to the part of the gene encoding the outer-membrane associated part of the protein, and 5′ to the peptidoglycan-associating site. It has been realised that for OmpA homologue proteins, such a stop codon should be placed 3′ to a codon encoding a conserved F residue (as indicated in FIGS. 1 and 2, and schematically in FIG. 3). This conserved F residue should be retained in order to ensure proper folding of the truncated protein in the outer membrane. C-terminal truncates of OmpA homologues (and genes encoding them) retaining this conserved F residue (the identity of which can readily be determined by comparison of a OmpA homologue to the sequence match-ups of FIGS. 1 and 2) is a further aspect of this invention.

When the region of the gene 3′ of the region encoding the peptidoglycan-associating site is to be retained (for instance if it encodes a protective epitope [for instance in the case of P5 from H. influenzae ]), the peptidoglycan-associating site may be engineered by 1, 2, 3, 4, 5 or more point mutations, or by deletion of amino acids (preferably 1, 2, 3, 4, 5, 7, 10, or 15 amino acids or the whole of the peptidoglycan-associating site) from the peptidoglycan-associating site, such that the peptidoglycan-binding activity of the protein is attenuated (reduced at least 2 fold, preferably removed entirely) to the desired level.

For the purposes of this invention ‘peptidoglycan-associating site’ means the region of a peptidoglycan-associating protein which can be aligned with the peptidoglycan-associating sites marked on FIGS. 1 & 2 (either the boxed or delineated regions).

The above down-regulation and mutation events on the bacterial genome may be carried out by the skilled person using homologous recombination (as described in the Examples and in WO 01/09350 incorporated by reference herein). For this technique, knowledge of at least 50-100 nucleotides (preferably around 500) either side of the area of change should be known.

Bacteria harbouring mutations (e.g. knock-outs) of the minB locus are not intended to be covered by this invention, unless the bacterium has also been modified by either or both of the above processes of the invention.

The hyperblebbing Gram-negative bacterium may be selected from the group consisting of any bacterium from the Neisseria family (for instance Neisseria meningitidis, Neisseria lactamica, Neisseria gonorrhoeae), Helicobacter pylori, Salmonella typhi, Salmonella typhimurium, Vibrio cholerae, Shigella spp., Haemophilus influenzae (particularly non-typeable), Bordetella pertussis, Pseudomonas aeruginosa and Moraxella catarrhalis.

Neisseria

In one embodiment the hyperblebbing Gram-negative bacterium is a Neisseria (preferably Neisseria meningitidis) strain which has been genetically modified by down-regulating expression of either or both of the following genes: exbB (homologous to tolQ) [SEQ ID NO:1] and exbD (homologous to tolR) [SEQ ID NO:3]. The upstream region of exbB and exbD is provided in SEQ ID NO:5 and 6, respectively, which is useful for designing homologous recombination vectors for down-regulating expression of the gene (for instance by deleting the promoter or replacing it with a weaker, or a metabolite-controlled promoter [e.g. the phoE promoter of E. coli ]).

In a further embodiment the hyperblebbing Neisseria (preferably Neisseria meningitidis) strain has been genetically modified (in isolation or in combination with the above down-regulation events) by mutation of rmpM [SEQ ID NO:7 or 9] to attenuate the peptidoglycan-binding activity of the encoded protein. The peptidoglycan-associating site for the protein can be seen in FIG. 1 (and has the amino acid sequence NQALSERRAYVVANNLVSN—see also SEQ ID NO:8). The upstream region of the gene is provided in SEQ ID NO:10 which is useful for the down-regulation of the gene. Preferably the gene is mutated in the way described in Example 1. If a truncate is made, it is preferred to introduce the stop codon downstream of the codon encoding the conserved F residue as indicated in FIGS. 1 and 2.

Vesicles prepared from such modifed strains may have one or more of the following improvements: reduced particle size (allowing sterile filtration through 0.22 μm pores), an increased batch homogeneity, and a superior yield. Such kind of alterations on bleb morphology are obtained by manipulating genes involved in linking the outer membrane to the peptidoglycan layer and/or to the cytoplasmic membrane as described above. Unproved, natural bleb shedding has the advantage that blebs may be isolated in industrial quantities without the use of detergents such as deoxycholate.

Haemophilus influenzae

In one embodiment the hyperblebbing Gram-negative bacterium is a Haemophilus influenzae (preferably non-typeable) strain which has been genetically modified by down-regulating expression of one or more of the following genes: tolQ [SEQ ID NO:11], tolR [SEQ ID NO:13], tolA [SEQ ID NO:15] and tolB [SEQ ID NO:17]. The genes are present in a single operon, and thus the upstream region provided in SEQ ID NO:19, is useful for designing homologous recombination vectors for down-regulating expression of all genes on the operon (for instance by deleting the promoter or replacing it with a weaker, or a metabolite controlled promoter [e.g. the phoE promoter of E. coli ]). Preferred embodiments include deleting both tolQ & R genes, or both tolR & A genes (preferably as described in Examples 4 and 5, respectively), whilst maintaining expression of the other genes on the operon (particularly tolB).

In a further embodiment the hyperblebbing Haemophilus influenzae (preferably non-typeable) strain has been genetically modified (in isolation or in combination with the above down-regulation events) by mutation of of one or more genes selected from a group consisting of: ompP5 [SEQ ID NO:20], ompP6 [SEQ ID NO:22 or 24] and pcp [SEQ ID NO:26] to attenuate the peptidoglycan-binding activity of the encoded protein. The peptidoglycan-associating site for the proteins can be seen in FIG. 1. Preferably the genes are mutated in a similar way to that described in Example 6. If a truncate is made of P5 or P6, it is preferred to introduce the stop codon downstream of the codon encoding the conserved F residue as indicated in FIG. 1.

For P5, the region of the gene 3′ of the region encoding the peptidoglycan-associating site may advantageously be retained (as it encodes a protective epitope). In such case, the peptidoglycan-associating site may be engineered by 1, 2, 3, 4, 5 or more point mutations, or by deletion of amino acids (preferably 1, 2, 3, 4, 5, 7, 10, or 15 amino acids, or the whole of the peptidoglycan-associating site) from the peptidoglycan-associating site, such that the peptidoglycan-binding activity of the protein is reduced (preferably, removed entirely) to the desired level, whilst retaining the protective epitope.

Preferred bacteria have down-regulated tolQ&R and mutated P5, or down-regulated tolR&A and mutated P5 phenotypes.

The P5 gene has been found to be homologous with E. coli OmpA gene, and the P6 gene has been found to be homologous with E. coli Pal gene (P5 and OmpA proteins are 51% identical, P6 and Pal proteins are 62% identical). The pcp gene (also called 1pp) encodes a lipoprotein similar neither to E coli Lpp nor to E coli Pal, but contains a peptidoglycan-associating site (FIG. 1).

Vesicles prepared from such modified strains may have one or more of the following improvements: reduced particle size (allowing sterile filtration through 0.22 μm pores), an increased batch homogeneity, and a superior yield. Such kind of alterations on bleb morphology are obtained by manipulating genes involved in linilng the outer membrane to the peptidoglycan layer and/or to the cytoplasmic membrane as described above. Improved, natural bleb shedding has the advantage that blebs may be isolated in industrial quantities without the use of detergents such as deoxycholate.

Moraxcella catarrhalis

In one embodiment the hyperblebbing Gram-negative bacterium is a Moraxella catarrhalis strain which has been genetically modified by down-regulating expression of one or more of the following genes: tolQ [SEQ ID NO:28], tolR [SEQ ID NO:30], tolX [SEQ ID NO:32], tolB [SEQ ID NO:34] and tolA [SEQ ID NO:36]. The to1QRXB genes are present in a single operon, and thus the upstream region provided upstream of SEQ ID NO:28, is useful for designing homologous recombination vectors for down-regulating expression of all genes on the operon (for instance by deleting the promoter or replacing it with a weaker, or a metabolite-controlled promoter [e.g. the phoE promoter of E. coli ]). Upstream sequence is also provided upstream of SEQ ID NO:36 for similarly doing so to the tolA gene. Preferred embodiments include deleting both tolQ & R genes, or both tolR & X genes (preferably as described in Example 2), whilst maintaining expression of the other genes on the operon (particularly tolB).

In a further embodiment the hyperblebbing Moraxella catarrhalis strain has been genetically modified (in isolation or in combination with the above down-regulation events) by mutation of of one or more genes selected from a group consisting of: ompCD [SEQ ID NO:38], xompA [SEQ ID NO:40; WO 00/71724], Pa11 [SEQ ID NO:42], and pal2 [SEQ ID NO:44], to attenuate the peptidoglycan-binding activity of the encoded protein. The peptidoglycan-associating site for the proteins can be seen in FIG. 2. Preferably the genes are mutated in a similar way to that described in Example 3. If a truncate is made of OQMPCD, XOMPA or Pa11 or Pa12, it is preferred to introduce the stop codon downstream of the codon encoding the conserved F residue as indicated in FIG. 2.

Preferred bacteria have down-regulated tolQ&R and mutated ompCD, or down-regulated tolR&X and mutated ompCD phenotypes.

The OMPCD gene has been found to be homologous with E. coli OmpA gene. The OmpCD encoded protein is not well conserved in its N-terminal domain, compared to OmpA. However, it contains a proline, alanine and valine rich “hinge” region and its C-terminal domain is significantly similar to the C-terminal domain of OmpA (25% identity in 147 aa overlap). Two genes encoding lipoproteins related to Pal have also been identified (Pa11 and Pal2 are respectivily 39% and 28% identical to E. coli Pal). These lipoproteins, as well as the C-terminal domain of OmpCD, contain a putative PGAS (FIG. 2). A fourth gene (xOmpA) encoding a protein containing a putative PGAS has been identified in M. catarrhalis. The N-terminal domain of this protein shows no significant similarity to any known protein. However, its C-terminal domain is similar to the C-terminal domain of OmpA (25% identity in 165 aa overlap) (FIG. 2).

Vesicles prepared from such modifed strains may have one or more of the following improvements: reduced particle size (allowing sterile filtration through 0.22 μm pores), an increased batch homogeneity, and a superior yield. Such kind of alterations on bleb morphology are obtained by manipulating genes involved in linking the outer membrane to the peptidoglycan layer and/or to the cytoplasmic membrane as described above. Improved, natural bleb shedding has the advantage that blebs may be isolated in industrial quantities without the use of detergents such as deoxycholate.

Further Improvements in the Bacteria and Blebs of the Invention

The hyperblebbing Gram-negative bacterium may be further genetically engineered by one or more processes selected from the following group: (a) a process of down-regulating expression of immunodominant variable or non-protective antigens, (b) a process of upregulating expression of protective OMP antigens, (c) a process of down-regulating a gene involved in rendering the lipid A portion of LPS toxic, (d) a process of upregulating a gene involved in rendering the lipid A portion of LPS less toxic, and (e) a process of down-regulating synthesis of an antigen which shares a structural similarity with a human structure and may be capable of inducing an auto-immune response in humans.

Such bleb vaccines of the invention are designed to focus the immune response on a few protective preferably conserved) antigens or epitopes—formulated in a multiple component vaccine. Where such antigens are integral OMPs, the outer membrane vesicles of bleb vaccines will ensure their proper folding. This invention provides methods to optimize the OMP and LPS composition of OMV (bleb) vaccines by deleting immunodominant variable as well as non protective OMPs, by creating conserved OMPs by deletion of variable regions, by upregulating expression of protective OMPs, and by eliminating control mechanisms for expression (such as iron restriction) of protective OMPs. In addition the invention provides for the reduction in toxicity of lipid A by modification of the lipid portion or by changing the phosphoryl composition whilst retaining its adjuvant activity or by masking it. Each of these new methods of improvement individually improve the bleb vaccine, however a combination of one or more of these methods work in conjunction so as to produce an optimised engineered bleb vaccine which is immuno-protective and non-toxic—particularly suitable for paediatric use.

(a) a Process of Down-Regulating Expression of Immunodominant Variable or Non-Protective Antigens

Many surface antigens are variable among bacterial strains and as a consequence are protective only against a limited set of closely related strains. An aspect of this invention covers the reduction in expression, or, preferably, the deletion of the gene(s) encoding variable surface protein(s) which results in a bacterial strain producing blebs which, when administered in a vaccine, have a stronger potential for cross-reactivity against various strains due to a higher influence exerted by conserved proteins (retained on the outer membranes) on the vaccinee's immune system. Examples of such variable antigens include: for Neisseria—pili (PilC) which undergoes antigenic variations, PorA, Opa, TbpB, FrpB; for H. influenzae—P2, P5, pilin, IgA1-protease; and for Moraxella—CopB, OMP106.

Other types of gene that could be down-regulated or switched off are genes which, in vivo, can easily be switched on (expressed) or off by the bacterium. As outer membrane proteins encoded by such genes are not always present on the bacteria, the presence of such proteins in the bleb preparations can also be detrimental to the effectiveness of the vaccine for the reasons stated above. A preferred example to down-regulate or delete is Neisseria Opc protein. Anti-Opc immunity induced by an Opc containing bleb vaccine would only have limited protective capacity as the infecting organism could easily become Opc⁻. H. influenzae HgpA and HgpB are other examples of such proteins.

In process a), these variable or non-protective genes are down-regulated in expression, or terminally switched off. This has the surprising advantage of concentrating the immune system on better antigens that are present in low amounts on the outer surface of blebs.

The strain can be engineered in this way by a number of strategies including transposon insertion to disrupt the coding region or promoter region of the gene, or point mutations or deletions to achieve a similar result. Homologous recombination may also be used to delete a gene from a chromosome (where sequence X comprises part (preferably all) of the coding sequence of the gene of interest). It may additionally be used to change its strong promoter for a weaker (or no) promoter. All these techniques are described in WO 01/09350 (published by WIPO on Aug. 2, 2001 and incorporated by reference herein).

(b) a Process of Upregulating Expression of Protective OMP Antigens

This may be done by inserting a copy of such a protective OMP into the genome (preferably by homologous recombination), or by upregulating expression of the native gene by replacing the native promoter for a stronger promoter, or inserting a strong promoter upstream of the gene in question (also by homologous recombination). Such methods can be accomplished using the techniques described in WO 01/09350 (published by WIPO on Aug. 2, 2001 and incorporated by reference herein).

Such methods are particularly useful for enhancing the production of immunologically relevant Bleb components such as outer-membrane proteins and lipoproteins (preferably conserved OMPs, usually present in blebs at low concentrations).

(c) a Process of Down-Regulating a Gene Involved in Rendering the Lipid A Portion of LPS Toxic

The toxicity of bleb vaccines presents one of the largest problems in the use of blebs in vaccines. A further aspect of the invention relates to methods of genetically detoxifying the LPS present in Blebs. Lipid A is the primary component of LPS responsible for cell activation. Many mutations in genes involved in this pathway lead to essential phenotypes. However, mutations in the genes responsible for the terminal modifications steps lead to temperature-sensitive (htrB) or permissive (msbB) phenotypes. Mutations resulting in a decreased (or no) expression of these genes result in altered toxic activity of lipid A. Indeed, the non-lauroylated (htrB mutant) [also defined by the resulting LPS lacking both secondary acyl chains] or non-myristoylated (msbB mutant) [also defined by the resulting LPS lacking only a single secondary acyl chain] lipid A are less toxic than the wild-type lipid A. Mutations in the lipid A 4′-kinase encoding gene (lpxK) also decreases the toxic activity of lipid A.

Process c) thus involves either the deletion of part (or preferably all) of one or more of the above open reading frames or promoters. Alternatively, the promoters could be replaced with weaker promoters. Preferably the homologous recombination techniques are used to carry out the process. Preferably the methods described in WO 01/09350 (published by WIPO on Aug. 2, 2001 and incorporated by reference herein) are used. The sequences of the htrB and msbB genes from Neisseria meningitidis B, Moraxella catarrhalis, and Haemophilus influenzae are provided in WO 01/09350 for this purpose.

(d) a Process of Upregulating a Gene Involved in Rendering the Lipid A Portion of LPS Less Toxic

LPS toxic activity could also be altered by introducing mutations in genes/loci involved in polymyxin B resistance (such resistance has been correlated with addition of aminoarabinose on the 4′ phosphate of lipid A). These genes/loci could be pmrE that encodes a UDP-glucose dehydrogenase, or a region of antimicrobial peptide-resistance genes common to many enterobacteriaciae which could be involved in aminoarabinose synthesis and transfer. The gene pmrF that is present in this region encodes a dolicol-phosphate manosyl transferase (Gunn J. S., Kheng, B. L., Krueger J., Kim K., Guo L., Hackett M., Miller S. I. 1998. Mol. Microbiol. 27:1171-1182).

Mutations in the PhoP-PhoQ regulatory system, which is a phospho-relay two component regulatory system (f. i. PhoP constitutive phenotype, PhoP^{c}), or low Mg⁺⁺ environmental or culture conditions (that activate the PhoP-PhoQ regulatory system) lead to the addition of aminoarabinose on the 4′-phosphate and 2-hydroxymyristate replacing myristate (hydroxylation of myristate). This modified lipid A displays reduced ability to stimulate E-selectin expression by human endothelial cells and INF-α secretion from human monocytes.

Process d) involves the upregulation of these genes using a strategy as described in WO 01/09350 (published by WIPO on Aug. 2, 2001 and incorporated by reference herein).

(e) a Process of Down-Regulating Synthesis of an Antigen Which Shares a Structural Similarity with a Human Structure and may be Capable of Inducing an Auto-Immune Response in Humans

The isolation of bacterial outer-membrane blebs from encapsulated Gram-negative bacteria often results in the co-purification of capsular polysaccharide. In some cases, this “contaminant” material may prove useful since polysaccharide may enhance the immune response conferred by other bleb components. In other cases however, the presence of contaminating polysaccharide material in bacterial bleb preparations may prove detrimental to the use of the blebs in a vaccine. For instance, it has been shown at least in the case of N. meningitidis that the serogroup B capsular polysaccharide does not confer protective immunity and is susceptible to induce an adverse auto-immune response in humans. Consequently, process e) of the invention is the engineering of the bacterial strain for bleb production such that it is free of capsular polysaccharide. The blebs will then be suitable for use in humans. A particularly preferred example of such a bleb preparation is one from N. meningitidis serogroup B devoid of capsular polysaccharide.

This may be achieved by using modified bleb production strains in which the genes necessary for capsular biosynthesis and/or export have been impaired as described in WO 01/09350 (published by WIPO on Aug. 2, 2001 and incorporated by reference herein). A preferred method is the deletion of some or all of the Neisseria meningitidis cps genes required for polysaccharide biosynthesis and export. For this purpose, the replacement plasmid pMF121 (described in Frosh et al. 1990, Mol. Microbiol. 4:1215-1218) can be used to deliver a mutation deleting the cpsCAD (+galE) gene cluster. Alternatively the siaD gene could be deleted, or down-regulated in expression (the meningococcal siaD gene encodes alpha-2,3-sialyltransferase, an enzyme required for capsular polysaccharide and LOS synthesis). Such mutations may also remove host-similar structures on the saccharide portion of the LPS of the bacteria.

Combinations of Methods a)-e)

It may be appreciated that one or more of the above processes may be used to produce a modified strain from which to make improved bleb preparations of the invention. Preferably one such process is used, more preferably two or more (2, 3, 4, or 5) of the processes are used in order to manufacture the bleb vaccine. As each additional method is used in the manufacture of the bleb vaccine, each improvement works in conjunction with the other methods used in order to make an optimised engineered bleb preparation.

A preferred meningococcal (particularly N. meningitidis B) bleb preparation comprises the use of processes b), c) and e) (optionally combined with process a)). Such bleb preparations are safe (no structures similar to host structures), non-toxic, and structured such that the host immune response will be focused on high levels of protective (and preferably conserved) antigens. All the above elements work together in order to provide an optimised bleb vaccine.

Similarly for M. catarrhalis, non-typeable H. influenzae, and non serotype B meningococcal strains (e.g. serotype A, C, Y or W), preferred bleb preparations comprise the use of processes b) and c), optionally combined with process a).

Preferred Neisserial Bleb Preparations

One or more of the following genes (encoding protective antigens) are preferred for upregulation via process b) when carried out on a Neisserial strain, including gonococcus, and meningococcus (particularly N. meningitidis B): NspA (WO 96/29412), Hsf-like (WO 99/31132), Hap (PCT/EP99/02766), PorA, PorB, OMP85 (WO 00/23595), PilQ (PCT/EP99/03603), PldA (PCT/EP99/06718), FrpB (WO 96/31618), ThpA (U.S. Pat. No. 5,912,336), TbpB, FrpA/FrpC (WO 92/01460), LbpA/LbpB (PCT/EP98/05117), FhaB (WO 98/02547), HasR (PCT/EP99/05989), lipo02 (PCT/EP99/08315), Thp2 (WO 99/57280), MltA (WO 99/57280), and ctrA (PCT/EP00/00135). They are also preferred as genes which may be heterologously introduced into other Gram-negative bacteria.

One or more of the following genes are preferred for downregulation via process a): PorA, PorB, PilC, ThpA, TbpB, LbpA, LbpB, Opa, and Opc (most preferably PorA).

One or more of the following genes are preferred for downregulation via process c): htrB, msbB and lpxK (most preferably msbB which removes only a single secondary acyl chain from the LPS molecule).

One or more of the following genes are preferred for upregulation via process d): pmrA, pmrB, pmrE, and pmrF.

One or more of the following genes are preferred for downregulation via process e): galE, siaA, siab, siaC, siaD, ctrA, ctrB, ctrC, and ctrD (the genes are described in described in WO 01/09350-published by WIPO on Aug. 2, 2001 and incorporated by reference herein).

Preferred Pseudomonas aeruginosa Bleb Preparations

One or more of the following genes (encoding protective antigens) are preferred for upregulation via process b): PcrV, OprF, OprI. They are also preferred as genes which may be heterologously introduced into other Gram-negative bacteria.

Preferred Moraxella catarrhalis Bleb Preparations

One or more of the following genes (encoding protective antigens) are preferred for upregulation via process b): OMP106 (WO 97/41731 & WO 96/34960), HasR (PCT/EP99/03824), PilQ (PCT/EP99/03823), OMP85 (PCT/EP00/01468), lipo06 (GB 9917977.2), lipo10 (GB 9918208.1), lipo11 (GB 9918302.2), lipo18 (GB 9918038.2), P6 (PCT/EP99/03038), ompCD, CopB (Helminen M E, et al (1993) Infect. Immun. 61:2003-2010), D15 (PCT/EP99/03822), Omp1A1 (PCT/EP99/06781), Hly3 (PCT/EP99/03257), LbpA and LbpB (WO 98/55606), ThpA and TbpB (WO 97/13785 & WO 97/32980), OmpE, UspA1 and UspA2 (WO 93/03761), FhaB (WO 99/58685) and Omp21. They are also preferred as genes which may be heterologously introduced into other Gram-negative bacteria

One or more of the following genes are preferred for downregulation via process a): CopB, OMP106, OmpB1, TbpA, TbpB, LbpA, and LbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Preferred Haemophilus influenzae Bleb Preparations

One or more of the following genes (encoding protective antigens) are preferred for upregulation via process b): D15 (WO 94/12641), P6 (EP 281673), ThpA, TbpB, P2, P5 (WO 94/26304), OMP26 (WO 97/01638), HMW1, HMW2, HMW3, HMW4, Hia, Hsf, Hap, Hin47, Iomp1457 (GB 0025493.8), YtfN (GB 0025488.8), VirG (GB 0026002.6), Iomp1681 (GB 0025998.6), OstA (GB 0025486.2) and Hif (all genes in this operon should be upregulated in order to upregulate pilin). They are also preferred as genes which may be heterologously introduced into other Gram-negative bacteria.

One or more of the following genes are preferred for downregulation via process a): P2, P5, Hif, IgA1-protease, HgpA, HgpB, HMW1, HMW2, Hxu, ThpA, and TbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Preparations of Membrane Vesicles (Blebs) of the Invention

The manufacture of bleb preparations from any of the aforementioned modified strains may be achieved by harvesting blebs naturally shed by the bacteria, or by any of the methods well known to a skilled person (e.g. as disclosed in EP 301992, U.S. Pat. No. 5,597,572, EP 11243 or U.S. Pat. No. 4,271,147).

A preparation of membrane vesicles obtained from the bacterium of the invention is a further aspect of this invention. Preferably, the preparation of membrane vesicles is capable of being filtered through a 0.22 μm membrane.

A sterile (preferably homogeneous) preparation of membrane vesicles obtainable by passing the membrane vesicles from the bacterium of the invention through a 0.22 μm membrane is also envisaged.

Vaccine Formulations

A vaccine which comprises a bacterium of the invention or a bleb preparation of the invention together with a pharmaceutically acceptable diluent or carrier is a further aspect of the invention. Such vaccines are advantageously used in a method of treatment of the human or animal body.

Vaccine preparation is generally described in Vaccine Design (“The subunit and adjuvant approach” (eds Powell M. F. & Newman M. J.) (1995) Plenum Press New York).

The vaccine preparations of the present invention may be adjuvantei Suitable adjuvants include an aluminium salt such as aluminum hydroxide gel (alum) or aluminium phosphate, but may also be a salt of calcium particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used include, Monophosphoryl lipid A, particularly 3-de-O-acylated monophosphoryl lipid A, and a combination of monophosphoryl lipid A, preferably 3-de-O-acylated monophosphoryl lipid A (3D-MPL) together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO96/33739. A particularly potent adjuvant formulation involving QS213D-MPL and tocopherol in an oil in water emulsion is described in WO95/17210 and is a preferred formulation.

The vaccine may comprise a saponin, more preferably QS21. It may also comprise an oil in water emulsion and tocopherol. Unmethylated CpG containing oligo nucleotides (WO 96/02555) are also preferential inducers of a TH1 response and are suitable for use in the present invention.

The vaccine preparation of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of protecting an individual against a bacterial infection which comprises administering to the individual an effective amount (capable of immunoprotecting an individual against the source bacterium) of a bacterium of the invention or a bleb preparation of the invention.

The amount of antigen in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-100 μg of protein antigen, preferably 5-50 μg, and most typically in the range 5-25 μg.

An optimal amount for a particular vaccine can be ascertained by standard studies involving observation of appropriate immune responses in subjects. Following an initial vaccination, subjects may receive one or several booster immunisations adequately spaced.

A process for preparing a vaccine composition comprising a preparation of membrane vesicles of the invention is also envisaged which process comprises: (a) inoculating a culture vessel containing a nutrient medium suitable for growth of the bacterium of the invention; (b) culturing said bacterium; (c) recovering membrane vesicles from the medium; and (d) mixing said membrane vesicles with a pharmaceutically acceptable diluent or carrier. The vesicles may be recovered by detergent (e.g. deoxycholate) extraction, but are preferably recovered without such a step (and necessary chromatography and ultracentrifugation steps that go with it)

Preferably after either step (c) or step (d), the prepartion is sterile-filtered (through a 0.22 μm membrane).

A method for producing a hyperblebbing bacterium or the invention is also provided, which method comprises genetically modifying a Gram-negative bacterial strain by either or both of the following processes: (a) engineering the strain to down-regulate expression of one or more Tol genes; and (b) mutating one or more gene(s) encoding a protein comprising a peptidoglycan-associated site to attenuate the peptidoglycan-binding activity of the protein(s).

Nucleotide Sequences of the Invention

A further aspect of the invention relates to the provision of nucleotide sequences (see appended sequence listings) which may be used in the processes (down-regulation/mutation) of the invention.

Another aspect of the invention is an isolated polynucleotide sequence which hybridises under highly stringent conditions to at least a 30 nucleotide portion of a nucleotide sequence of the invention (e.g. SEQ ID NO:1, 3, 5, 6, 7, 9, 10, 11, 13, 15, 17, 19, 20, 22, 24, 26, 28, 30, 32, 34, 36, 38, 40, 42, or 44) or a complementary strand thereof. Preferably the isolated sequence should be long enough to perform homologous recombination with the chromosomal sequence if it is part of a suitable vector—namely at least 30 nucleotides (preferably at least 40, 50, 60, 70, 80, 90, 100, 200, 300, 400, or 500 nucleotides). More preferably the isolated polynucleotide should comprise at least 30 nucleotides (preferably at least 40, 50, 60, 70, 80, 90, 100, 200, 300, 400, or 500 nucleotides) of the actual sequences provided or a complementary strand thereof.

As used herein, highly stringent hybridization conditions include, for example, 6×SSC, 5× Denhardt, 0.5% SDS, and 100 μg/mL fragmented and denatured salmon sperm DNA hybridized overnight at 65° C. and washed in 2×SSC, 0.1% SDS one time at room temperature for about 10 minutes followed by one time at 65° C. for about 15 minutes followed by at least one wash in 0.2×SCC, 0.1% SDS at room temperature for at least 3-5 minutes.

A further aspect is the use of the isolated polynucleotide sequences of the invention in performing a genetic engineering event (such as transposon insertion, or site specific mutation or deletion, but preferably a homologous recombination event) within a Gram-negative bacterial chromosomal gene in order to down-regulate or mutate it as described above. Preferably the strain in which the recombination event is to take place is the same as the strain from which the sequences of the invention were obtained. However, the meningococcus A, B, C, Y and W and gonococcus genomes are sufficiently similar that sequence from any of these strains may be suitable for designing vectors for performing such events in the other strains. This is likely also to be the case for Haemophilus influenzae and non-typeable Haemophilus influenzae.

Cited documents are incorporated by reference herein.

### EXAMPLES

The examples below are carried out using standard techniques, which are well known and routine to those of skill in the art, except where otherwise described in detail. The examples are illustrative, but do not limit the invention.

### Example 1

Construction of a Neisseria meningitidis Strain Lacking Functional RmpM Gene

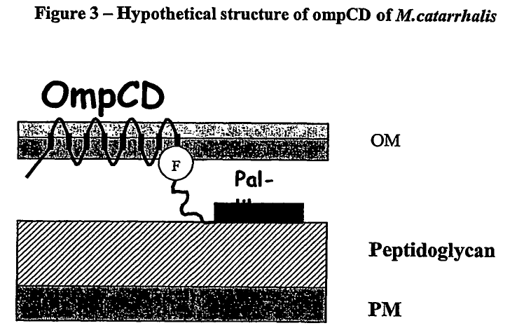

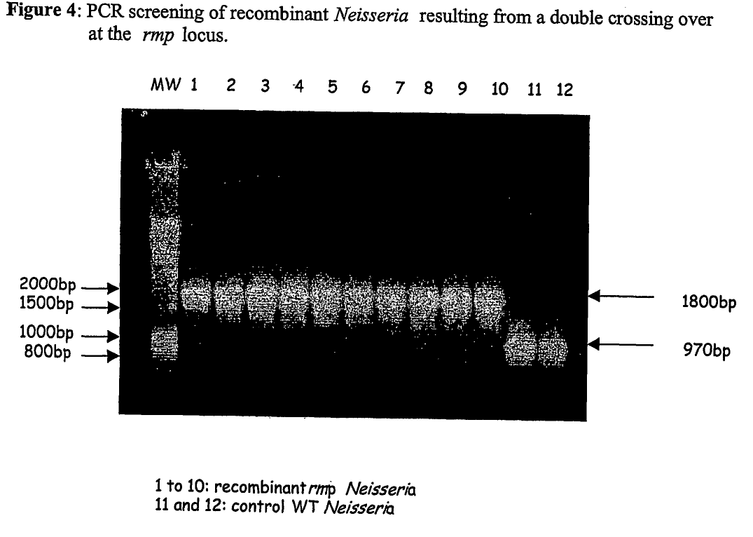

The aim of the experiment was to construct a Neisseria meningitidis serogroup B strain expressing a truncated Rmp protein. Neisseria meningitidis Rmp is homologous to E. coli OmpA and P. aeruginosa OprF. This protein contains an N-terminal domain anchored in the external membrane, and a C-terminal domain containing a peptidoglycan associated site. The C-terminal domain of Rmp was deleted by homologous recombination in a Neisseria meningitidis serogroup B cps-strain. The expressed N-terminal part of the protein will still play its role in the stability of the external membrane, while the absence of the peptidoglycan associated site will relax the membrane around the bacterium. Outer membrane vesicles from this modified Neisseria were analyzed: amount of production, size, homogeneity. A DNA region (729bp) corresponding to the rmp gene was discovered (SEQ ID No 9) in the Sanger database containing genomic DNA sequences of the Neisseria meningitidis serogroup A strain Z2491. A similar sequence is present in Neisseria meningitidis serogroup B strain MC58 (SEQ ID No 7); it shows 99.3% identity with the men A sequence. A DNA fragment covering the complete sequence of the gene was PCR amplified from Neisseria meningitidis serogroup B genomic DNA, using oligonucleotides RMP-H-5 (5′-GCC CAC AAG CTT ATG ACC AAA CAG CTG AAA TT-3′) & RMP-E-3 (5′-CCG GAA TTC TTA GTG TTG GTG ATG ATT GT-3′) containing HindIII and EcoRI restriction sites (underlined). This PCR fragment was cleaned with a High Pure Kit (Roche, Mannheim, Germany) and directly cloned in a pGemT vector (Promega, USA). This plasmid was submitted to circle PCR mutagenesis (Jones & Winistofer (1992), Biotechniques 12: 528-534) in order to introduce a 33bp deletion and a stop codon after the internal phenylalanine residue. The circle PCR was performed using the oligonucleotides RMP-CIRC-3-B (5′-GGC GGA TCC TTA GAA CAG GGT TTT GGC AG-3′) & RMP CIRC-5-B (5′-CGG GGA TCC CAA GAC AAC CTG AAA GTA TT-3′) containing BamHI restriction sites (underlined). The cmR gene was amplified from pGPS2 plasmid, with oligonucleotides CM/BAM/5/2 (5′-CGC GGA TCC GCC GTC TGA AAC CTG TGA CGG AAG ATC AC-3′) & CM/BAM/3/2 (5′-CGC GGA TCC TTC AGA CGG CCC AGG CGT TTA AGG GCA C-3′) containing uptake sequences and BamHI restriction sites (underlined). This fragment was inserted in the circle PCR plasmid restricted with BamHI. The recombinant plasmid was used to transform Neisseria meningitidis serogroup B cps-strain. Recombinant Neisseria meningitidis resulting from a double crossing over event were selected by PCR screening with primers RMP SCR 5(5′-CAT GAT AGA CTA TCA GGA AAC-3′) and RMP SCR 3 (5′-CAG TAC CTG GTACAA AAT CC-3′). Those primers amplify a fragment of 970 bp from the control strain (WT for rmp) and one of 1800bp from the recombinant Neisseria. FIG. 4 shows the PCR amplifications obtained from 10 recombinant colonies analyzed on a 1% agarose gel in the presence of ethidium bromide. Recombinants were grown on GC medium containing 5 μg/ml chlorarnphenicol and analyzed for Rmp expression and OMV production.

Characterization of menB OMV's Produced From an rmpM Mutant

The effect of the rmpM mutation on OMV's yield, size and polydispersity was analyzed by comparing OMV's extracted (using Deoxycholate) from parental H44/76 Cps-(no capsular polysaccharide) and the corresponding OMV's extracted from the RmpM mutant derivative. The results are the following:

OMV's yields observed with different N. meningitidis H44/76
derivative strains grown in 400 ml Flask cultures

| | | |
|---|---|---|
| Strain Nm B1390 cps(−) porA(+) PilQ atg: | 2.7 | mg |
| Strain Nm B1391 cps(−) porA(−) PilQ atg: | 9.1 | mg |
| Strain B1405 cps(−) porA(−) RmpM (−): | 20 | mg |

As shown below, deletion of rmpM significantly increase (at least a factor 2) the yield of OMV's prepared from such a strain. The size of OMV's isolated from wild-type and rmpM mutants N. meningitidis H44/46 derivative strains was estimated by Photon Correlation Spectroscopy (PCS) using the Malvern Zetasizer 4000 analyzer as recommended by the supplier (Malvern Instruments GmbH, Herrenberg Germany: www.malvem.co.uk). Results are summarized below:

| | Z average | |
|---|---|---|
| Samples | diameter (nm) | Polydispersity |
| CPS (−) 07/2000 7.8 mg/ml | 136 | 0.31 |
| CPS (−) 09/2000 5.7 mg/ml | 166 | 0.42 |
| CPS (−)rmpM (−) B1405 6.7 mg/ml | 202 | 0.53 |

These data support that the size of CPS (−) 07/2000 is smaller than the size of CPS (−) 09/2000 and also that the size of CPS (−) samples is smaller than the size of CPS (−) rmpM (−) blebs. Altogether, these data support that deletion of a domain encoding the peptidoglycan associated domain of RmpM leads to enhanced blebbing and altered OMV morphology and size distribution. These features could be advantageously used for the production of vaccines as documented in WO 01/09350 (published by WIPO on Aug. 2, 2001 and incorporated by reference herein).

### Example 2

### Deletion of the tolQR Genes in Moraxella catarrhalis

The aim of the experiment was to delete the tolQR genes from Moraxella catarrhalis in order to obtain a hyperblebbing Moraxella strain.

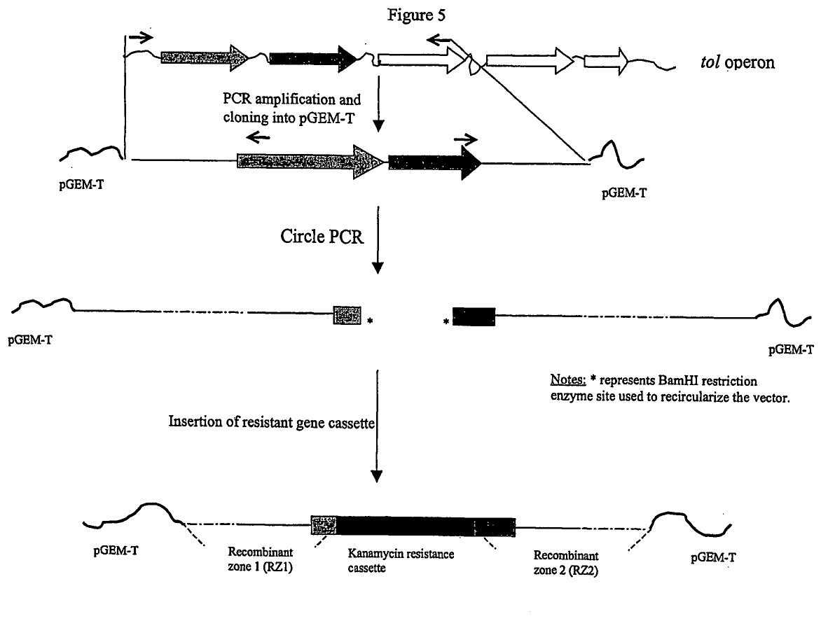

For that purpose, a mutator plasmid was constructed using E. coli cloning technologies. The main steps are shown in FIG. 5. Briefly, genomic DNA was extracted from the Moraxella catarrhalis strain ATCC 43617 using the QIAGEN genomic DNA extraction kit (Qiagen Gmbh). This material was used to amplify by polymerase chain reaction (PCR) a 2151 nucleotide-DNA fragment covering 501 nucleotides upstream of the tolQ gene start codon (ATG) to 500 nucleotides downstream of the tolR stop codon (TAA) using primers A (5′-GCTCTAGAGCTTCAGCAGTCACGGGCAAATCATGATTA-3′) and B (5′-CGGAGCTCTGCTCAAGGTCTGAGACATGATTAGAATAT-3′). This PCR product was introduced into the pGEM-T-cloning vector (Promega) according to the manufacturer's instructions. The obtained plasmid was then submitted to circle PCR mutagenesis (Jones and Winistofer, (1992), Biotechniques 12: 528-534) in order to delete the tol QR genes (consisting of an amplification of the entire vector without the region comprised between the two primers). The circle PCR was performed using primers C (5′-CGGGATCCCAGCGAGATTAGGCTAATGGATTCGTTCA-3′) and D (5′-CGGGATCCAATGTTGGTATCACCCAAGTGAGTTTGCTT-3′) hybridizing 31 nucleotides downstream of the start codon (ATG) of tolQ and 48 bp upstream of the stop codon (TAA) of tolR, respectively (see FIG. 5). Both primers contain a BamHI restriction site (underlined). The obtained PCR fragment was then purified using the PCR Clean Up Kit (Boehringer), digested by BamHI and ligated resulting in a plasmid carrying a 532 nucleotide-5′ flanking sequence and a 548 nucleotide-3′ flanking sequence separated by a BamHI restriction site. Kanamycin resistance cassettes were then introduced into the BamHI site in order to be able to select recombinants in the host bacteria Two different cassettes were subcloned giving two different plasmids, one was the kanamycin resistance gene from Tn903 (KanR) subcloned from plasmid pUC4K (Amersham Pharmacia Biotech) and the other was a sacB-neo cassette originating from pIB3279 carrying the kanamycin resistance gene from Tn5 and the sacB gene (Blomfield et al., (1991), Molecular Microbiology, 5: 1447-1457) sacB is a counter-selection marker deleterious for bacteria in the presence of sucrose and allows further pushing-out of the cassette. Both cassettes were subcloned using the available BamHI restriction sites. The sequences of the obtained clones have been confirmed using Big Dye Cycle Sequencing kit (Perkin Elmer) and an ABI 373A/PRISM DNA sequencer. Alternatively, the pKNG101 suicide vector can be used to introduce the mutation after subcloning the flanking regions into the multi-cloning site of the vector (Kaniga et al., (1991), Gene 109:137-141).

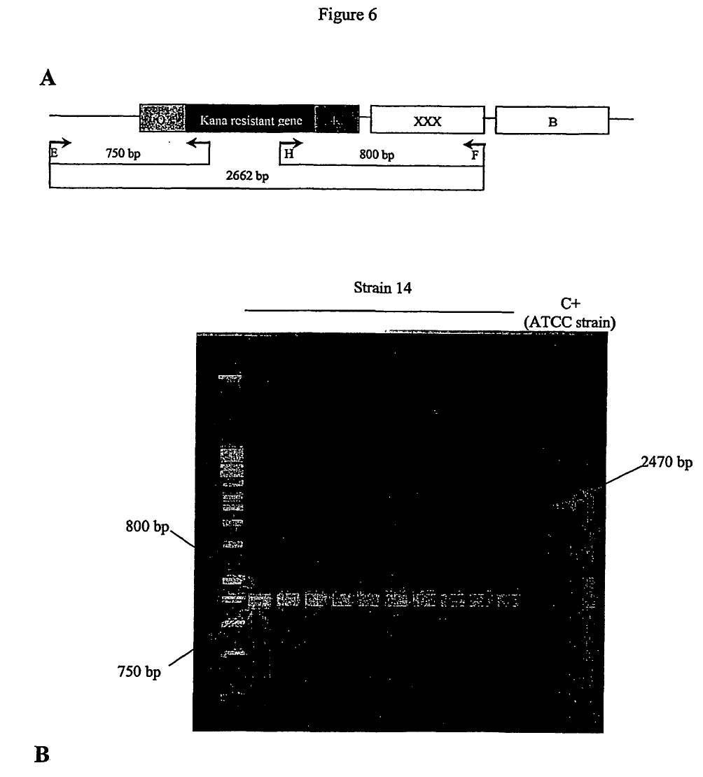

The plasmid carrying the kanamycin resistance marker from Tn903 was used to transform Moraxella catarrhalis strain 14 isolated from human nasopharynx in Oslo, Norway. The transformation technique is based on the natural DNA uptake competence of the strain. 10 bacterial colonies were mixed with 25 μg of DNA (in 20 μl PBS) and incubated for three hours at 36° C. Recombinant Moraxella catarrhalis clones were then selected on Muller-Hinton plates containing 20 μg/ml kanamycin and mutants resulting from a double recombinant event were screened by PCR using primers E (5′-ATCGGCGTGGCTGTGTGTGGC-3′), F (5′-ACCGAATTGGATTGAGGTCAC-3′), G (5′-GCGATTCAGGCCTGGTATGAG -3′) and H (5′-TTGTGCAATGTAACATCAGAG-3′). Following thermal amplification, a ˜10 μl aliquot of the reaction was analyzed by agarose gel electrophoresis (1% agarose in a Tris-borate-EDTA (TBE) buffer). DNA fragments were visualized by UV illumination after gel electrophoresis and ethidium bromide staining. A DNA molecular size standard (Smartladder, Eurogentec) was electophoresed in parallel with the test samples and was used to estimate the size of the PCR products. As shown in FIG. 6, several transformants produced the expected size PCR product and were identified as tolQR Moraxella catarrhalis mutant strains. Sequencing confirmed correct integration of the cassette. These clones can be tested for outer membrane vesicles production.

### Example 3

### Mutation of ompCD from Moraxella catarrhalis

The aim of the experiment was to mutate the ompCD gene from Moraxella catarrhalis into a truncated gene without the peptidoglycan-associated 3′-coding region in order to obtain a hyperblebbing Moraxella strain. In this experiment, a stop codon was introduced after the phenylalanine at the end of the transmembrane domain of the protein.

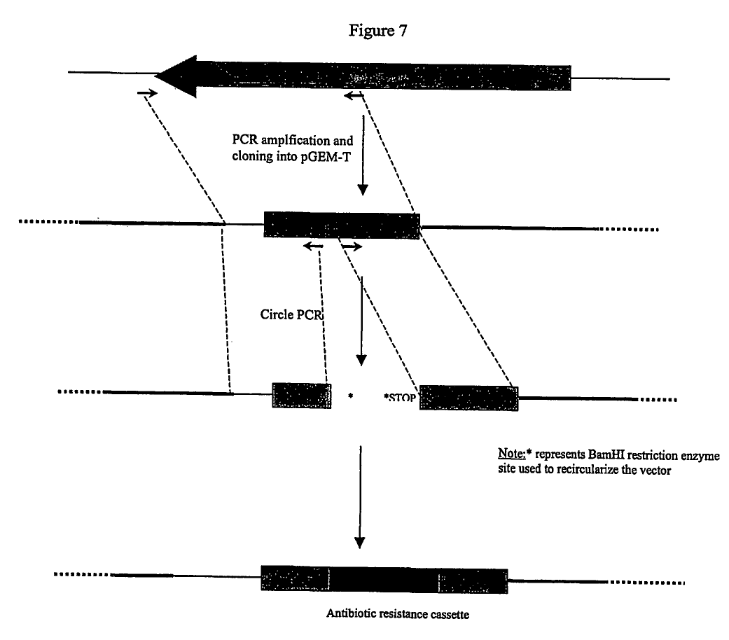

For that purpose, a mutator plasmid was constructed using E. coli cloning technologies. The main steps are shown in FIG. 7. Briefly, genomic DNA was extracted from the Moraxella catarrhalis strain ATCC 43617 using the QIAGEN genomic DNA extraction kit (Qiagen Gmbh). This material was used to amplify by polymerase chain reaction (PCR) a 1000 nucleotide-DNA fragment covering 500 nucleotides upstream and downstream of the critical phenylalanine residue, using primers 1 (5′-CCTCTAGACGCTTATTATAACATAAATCAGTCTAACTG-3′) and 2 (5′-AAGGTACCAGCAGAAGTAGCCAATGGGCAAAACATTGC-3′). This PCR product was introduced into the pGEM-T cloning vector (Promega) according to the manufacturer's instructions. The obtained plasmid was then submitted to circle PCR mutagenesis (Jones and Winistofer, (1992), Biotechniques 12: 528-534) in order to introduce a stop codon and a BamHI restriction site. The circle PCR was performed using primers 3 (5′-CCGGATCCTAACGGTATTGTGGTTTGATGATTGATTT-3′) and 4 (5′-AAGGATCCGCGCAAATGCGTGAATTCCCAAATGCAACT-3′) hybridizing 62 nucleotides upstream and 39 nucleotides downstream the TTC codon encoding the phenylalanine (FIG. 7). Both primers contain a BamHI restriction site (underlined) and primer 3 also contains the stop codon (bold). The obtained PCR fragment was then purified using the PCR Clean Up Kit (Boehringer), digested by BamHI and ligated resulting in a plasmid carrying a 438 nucleotide-5′ flanking sequence and 540 nucleotide-3′ flanking sequence separated by a BamHI site. Kanamycin resistance cassettes were then introduced into the BamHI site in order to be able to select recombinants in the host bacteria. Two different cassettes were subcloned giving two different plasmids, one was the kanamycin resistance gene from Tn903 (KanR) subcloned from plasmid pUC4K (Amersham Pharmacia Biotech) and the other was a SacB-neo cassette originating from pIB179 carrying the kanamycin resistance gene from Tn5 and the sacB gene (Blomfield et al., (1991), Molecular Microbiology, 5: 1447-1457). sacB is a counter-selection marker deleterious for bacteria in the presence of sucrose and allows further pushing-out of the cassette. Both cassettes were subcloned using the available BamHI restrictions sites. The sequences of the obtained clones were confirmed using Big Dye Sequencing kit (Perkin Elmer) and an ABI 373A/PRISM DNA sequencer. Alternatively, the pKNG101 suicide vector can be used to introduce the mutation after subcloning the flanking regions into the multi-cloning site of the vector (Kaniga et al., (1991), Gene 109:137-141).

The plasmid carrying the kanamycin resistance marker from Tn903 can be used to transform Moraxella catarrhalis. Recombinant Moraxella catarrhalis clones can be selected on Muller-Hinton plates containing 20 μg/ml kanamycin and mutants resulting from a double recombinant event can be screened by PCR. These clones can then be tested for outer membrane vesicles production.

### Example 4

### Deletion of the tolQR genes in non-typeable Haemophilus influenzae

The aim of the experiment was to delete the tolQR genes from non-typeable Haemophilus influenzae (NTHI) in order to obtain a hyperblebbing strain.

For that purpose, a mutator plasmid was constructed using E. coli cloning technologies. The main steps are shown in FIG. 5. Briefly, genomic DNA was extracted from the non-typeable Haemophilus influenzae strain 3224A using the QIAGEN genomic DNA extraction kit (Qiagen Gmbh). This material was used to amplify by polymerase chain reaction (PCR) a 1746 nucleotide-DNA fragment covering 206 nucleotides upstream of the tolQ gene codon to 364 nucleotides downstream of the tolR stop codon using primers ZR1-EcoRI (5′-CCGGAATTCAAAGTGCGGTAGATTTAGTCGTAGTAATTGATTTACTTATG-3′) and ZR2-XbaI (5′-CTAGTCTAGAACGTTGCTGTTCTTGCTG-3′). This PCR product was introduced into the pGEM-T cloning vector (Promega) according to the manufacturer's instructions. The obtained plasmid was then submitted to circle PCR mutagenesis (Jones and Winistofer, (1992), Biotechniques 12: 528-534) in order to delete the tol QR genes (consisting of an amplification of the entire vector without the region comprised between the two primers). The circle PCR was performed using primers ZR1-BamHI (5′-CGCGGATCCCGCTTCAGGTGCATCTGG-3′) and ZR2-BamHI (5′-CGCGGATCCAGACAGGAATTTGATAAGG-3′) hybridizing 312 nucleotides downstream of the start codon of tolQ and 144 bp upstream of the stop codon of tolR, respectively (FIG. 5). Both primers contain a BamHI restriction site (underlined). The obtained PCR fragment was then purified using the PCR Clean Up Kit (Boehringer), digested by BamHI and ligated resulting in a plasmid carrying a 517 nucleotide-5′ flanking sequence and a 507 nucleotide-3′ flanking region separated by a BamHI restriction site. Kanamycin resistance cassettes were then introduced into the BamHI site in order to be able to select recombinants in the host bacteria. Two different cassettes were subcloned giving two different plasmids, one was the kanamycin resistance gene from Tn903 (KanR) subcloned from plasmid pUC4K (Amersham Pharmacia Biotech) and the other was a sacB-neo cassette originating from pIB279 carrying the kanamycin resistance gene from Tn5 and the sacB gene (Blomfield et al., (1991), Molecular Microbiology, 5: 1447-1457). sacB is a counter-selection marker deleterious for bacteria in the presence of sucrose and allows further pushing-out of the cassette. Both cassettes were subcloned using the available BamHI restriction sites. The sequences of the obtained clones have been confirmed using Big Dye Cycle Sequencing kit Perkin Elmer) and an ABI 373A/PRISM DNA sequencer. Alternatively, the pKNG101 suicide vector can be used to introduce the mutation after subcloning the flanking regions into the multi-cloning site of the vector (Kaniga et al., (1991), Gene 109:137-141).

The plasmid carrying the kanamycin resistance marker from Tn903 was used to transform non-typeable Haemophilus influenzae strain 3224A. Transformation was realized using competent NTHI cells obtained by a calcium chloride treatment according to Methods in Enzymology, Bacterial genetic systems, ed. J. H. Miller, Academic Press Inc., vol. 204, p. 334. Recombinant non-typeable Haemophilus influenzae clones were selected on GC plates containing 15 μg/ml kanamycin and mutants resulting from a double recombinant event were screened by PCR using primers NTHI-Fo-ZR1 (5′-CCTTACTAGAGGAACAACAACTC-3′), NTHI-RE-ZR2 (5′-GCCTCTTCAGCTTGCTTCTG-3′), ZR1-EcoRI (5′-CCGGAATTCAAAGTGCGGTAGATTTAGTCGTAGTAATTGATTTACTTATG-3′) and ZR2-XbaI (5′-CTAGTCTAGAACGTTGCTGTTCTTGCTG-3′). Following thermal amplification, a 10 μl aliquot of the reaction was analyzed by agarose gel electrophoresis (1% agarose in a Tris-borate-EDTA (TBE) buffer). DNA fragments were visualized by UV illumination after gel electrophoresis and ethidium bromide staining. A DNA molecular size standard. (Smartladder, Eurogentec) was electrophoresed in parallel with the test samples and was used to estimate the size of the PCR products. Several transformants produced the expected size PCR product and were identified as non-typeable Haemophilus influenzae mutant strains carrying the antibiotic resistance cassette.

### Example 5

### Deletion of the toRA Genes in Non-Typeable Haemophilus influienzae

The aim of the experiment was to delete the tolRA genes from non-typeable Haemophilus influenzae (NTHI) in order to obtain a hyperblebbing strain.

For that purpose, a mutator plasmid was constructed using E. coli cloning technologies. The main steps are shown in FIG. 5. Briefly, genomic DNA was extracted from the non-typeable Haemophilus influenzae strain 3224A using the QIAGEN genomic DNA extraction kit (Qiagen Gmbh). This material was used to amplify by polymerase chain reaction (PCR) a 1797 nucleotide-DNA fragment covering 244 nucleotides upstream of the tolR gene codon to the tolA stop codon using primers ZR5-EcoRI (5′-CCGGAATTCAAAGTGCGGTAGATTTAGTCGTAATTCGCTGAGGCC-3′) and ZR6-XbaI (5′-CTAGTCTAGATTATCGAATATCAAAGTCAATAATG-3′). This PCR product was introduced into the pGEM-T cloning vector (Promega) according to the manufacturer's instructions. The obtained plasmid was then submitted to circle PCR mutagenesis (Jones and Winistofer, (1992), Biotechniques 12: 528-534) in order to delete the tolRA genes (consisting of an amplification of the entire vector without the region comprised between the two primers). The circle PCR was performed using primers ZR5-BamHI (5′ CGCGGATCCTTCTTCTGTTTAAACCTTCTTG-3′) and ZR6-BamHI (5′-CGCGGATCCAAGCAAAGGCTGAAGCGG-3′) hybridizing 257 nucleotides downstream of the start codon of tolR and 500 nucleotides upstream of the stop codon of tolA, respectively (see FIG. 5). Both primers contain a BamHI restriction site (underlined). The obtained PCR fragment was then purified using the PCR Clean Up Kit (Boehringer), digested by BamHI and ligated resulting in a plasmid carrying a 502 nucleotide-5′ flanking sequence and a 500 nucleotide-3′ flanking sequence separated by a BamHI restriction site. Kanamycin resistance cassettes were then introduced into the BamHI site in order to be able to select recombinants in the host bacteria. Two different cassettes were subcloned giving two different plasmids, one was the kanamycin resistance gene from Tn903 (KanR) subcloned from plasmid pUC4K (Amersham Pharmacia Biotehc) and the other was a sacB-neo cassette originating from pIB279 carrying the kanamycin resistance gene from Tn5 and the sacB gene (Blomfield et al., (1991), Molecular Microbiology, 5: 1447-1457). sacB is a counter-selection marker deleterious for bacteria in the presence of sucrose and allows further pushing-out of the cassette. Both cassettes were subcloned using the available BamHI restriction sites. The sequences of the obtained clones have been confirmed using Big Dye Cycle Sequencing kit (Perkin Elmer) and an ABI 373A/PRISM DNA sequencer. Alternatively, the pKNG101 suicide vector can be used to introduce the mutation after subcloning the flanking regions into the multi-cloning site of the vector (Kaniga et al., (1991), Gene 109:137-141).

The plasmid carrying the kanamycin resistance marker from Tn903 was used to transform non-typeable Haemophilus influenzae strain 3224. Transformation was realized using competent NTHI cells obtained by a calcium chloride treatment according to Methods in Enzymology, Bacterial genetic systems, ed. J. H. Miller, Academic Press Inc., vol. 204, p. 334. Recombinant non-typeable Haemophilus influenzae clones were selected on GC plates containing 15 μg/ml kanamycin and mutants resulting from a double recombinant event were screened by PCR using primers NTHI-FO-ZR5 (5′-CGCTGAGGCCTTGATTGC-3′), NTHI-RE-ZR6 (5′ -GTACAATCGCGAATACGCTCAC-3′), ZR5-EcoRI (5′-CCGGAATTCAAAGTGCGGTAGATTTAGTCGTAATTCGCTGAGGCC-3′) and ZR6-XbaI (5′-CTAGTCTAGATTATCGAATATCAAAGTCAATAATG-3′). Following thermal amplification, a ˜10 μl aliquot of the reaction was analyzed by agarose gel electrophoresis (1% agarose in a Tris-borate-EDTA (TBE) buffer). DNA fragments were visualized by UV illumination after gel electrophoresis and ethidium bromide staining. A DNA molecular size standard (Smartladder, Eurogentec) was electrophoresed in parallel with the test samples and was used to estimate the size of the PCR products. Several transformants produced the expected size PCR product and were identified as non-typeable Haemophilus influenzae mutant strains carrying the antibiotic resistance cassette.

### Example 6

### Mutation of P5 Gene in Non-Typeable Haemophilus influenzae

The aim of the experiment was to mutate the P5 gene from Haemophilus influenzae (NTHI) into a truncated gene without the peptidoglycan-associated 3′-coding region in order to obtain a hyperblebbing NTHI strain. In this experiment, a stop codon was introduced after the phenylalanine at the end of the transmembrane domain of the protein.

For that purpose, a mutator plasmid was constructed using E. coli cloning technologies. The main steps are shown in FIG. 7. Briefly, genomic DNA was extracted from the non-typeable Haemophilus influenzae strain 3224A using the QIAGEN genomic DNA extraction kit (Qiagen Gmbh). This material was used to amplify by polymerase chain reaction (PCR) a 1047 nucleotide-DNA fragment upstream and downstream of the TTT codon encoding the critical phenylalanine residue, using primers P5-01 bis (5′-GATGAATTCAAAGTGCGGTAGATTTAGTCGTAGTAATTAATAACTTA-3′) and P5-02 (5′-CTAGTCTAGAAGGTTTCCATAATGTTTCCTA-3′). This PCR product was introduced into the pGEM-T cloning vector (Promega) according to the manufacturer's instructions. The obtained plasmid was then submitted to circle PCR mutagenesis (Jones and Winistofer, (1992), Biotechniques 12: 528-534) in order to introduce a stop codon and a BamHI restriction site. The circle PCR was performed using primers P5-03 (5′-CGCGGATCCCTAAAAAGTTACATCAGAATTTAAGC -3′) and P5-04 (5′-CGCGGATCCGCATTTGGTAAAGCAAACTT-3′) hybridizing exactly at the TTT codon encoding the phenylalanine (see FIG. 7). Both primers contain a BamHI restriction site (underlined) and primer 3 also contains the stop codon (bold). The obtained PCR fragment was then purified using the PCR Clean Up Kit (Boehring), digested by BamHI and ligated resulting in a plasmid carrying a 518 nucleotide-5′ flanking sequence and a 538 nucleotide-3′ flanking sequence separated by a BamHI restriction site. Kanamycin resistance cassettes were then introduced into the BamHI site in order to be able to select recombinants in the host bacteria Two different cassettes were subcloned giving two different plasmids, one was the kanamycin resistance gene from Tn903 (KanR) subcloned from plasmid pUC4K (Amersham Pharmacia Biotech) and the other was a sacB-neo cassette originating from pIB279 carrying the kanamycin resistance gene from Tn5 and the sacB gene (Blomfield et al., (1991), Molecular Microbiology, 5: 1447-1457). sacB is a counter-selection marker deleterious for bacteria in the presence of sucrose and allows further pushing-out of the cassette. Both cassettes were subcloned using the available BamHI restriction sites. The sequences of the obtained clones were confirmed using Big Dye Cycle Sequencing kit (Perkin Elmer) and an ABI 373A/PRISM DNA sequencer. Alternatively, the pKNG101 suicide vector can be used to introduce the mutation after subcloning the flanking regions into the multi-cloning site of the vector (Kaniga et al., (1991), Gene 109:137-141).

The plasmid carrying the kanamycin resistance marker from Tn903 was used to transform non-typeable Haemophilus influenzae strain 3224. Transformation was realized using competent NTHI cells obtained by a calcium chloride treatment according to Methods in Enzymology, Bacterial genetic systems, ed. J. H. Miller, Academic Press Inc., vol. 204, p. 334. Recombinant non-typeable Haemophilus influenzae clones were selected on GC plates containing 15 μg/ml kanamycin and mutants resulting from a double recombinant event were screened by PCR using primers P5-01 bis (5′-GATGAATTCAAAGTGCGGTAGATTTAGTCGTAGTAATTAATAACTTA-3′) and P5-02 (5′-CTAGTCTAGAAGGTTTCCATAATGTITCCTA-3′). Following thermal amplification, a ˜10 μl aliquot of the reaction was analyzed by agarose gel electrophoresis (1% agarose in a Tris-borate-EDTA (TBE) buffer). DNA fragments were visualized by UV illumination after gel electrophoresis and ethidium bromide staining. A DNA molecular size standard (Smartladder, Eurogentec) was electrophoresed in parallel with the test samples and was used to estimate the size of the PCR products. Several transformants produced the expected size PCR product and were identified as non-typeable Haemophilus influenzae mutant strains carrying the antibiotic resistance cassette.

1. A hyperblebbing Gram-negative bacterium which has been genetically modified by one or more processes selected from a group consisting of: down-regulating expression of one or more Tol genes; and attenuating the peptidoglycan-binding activity by mutation of one or more gene(s) encoding a protein comprising a peptidoglycan-associated site.

2. The hyperblebbing Gram-negative bacterium of claim 1 which is selected from the group consisting of Neisseria meningitidis, Neisseria lactamica, Neisseria gonorrhoeae, Helicobacter pylori, Salmonella typhi, Salmonella typhimurium, Vibrio cholerae, Shigella spp., Haemophilus influenzae, Bordetelia pertussis, Pseudomonas aeruginosa and Moraxella catarrhalis.

3. The hyperblebbing Gram-negative bacterium of claim 2 which is a Neisseria meningitidis strain which has been genetically modified by down-regulating expression of either or both of the genes selected from a group consisting of: exbB (tolQ) and exbD (tolR).

4. The hyperblebbing Gram-negative bacterium of claim 2 or 3 which is a Neisseria meningitidis strain which has been genetically modified by mutation of rmpM to attenuate the peptidoglycan-binding activity of the encoded protein.

5. The hyperblebbing Gram-negative bacterium of claim 2 which is a Haemophilus influenzae strain which has been genetically modified by down-regulating expression of one or more genes selected from a group consisting of: tolQ, tolR, tolA and tolB.

6. The hyperblebbing Gram-negative bacterium of claim 2 or 5 which is a Haemophilus influenzae strain which has been genetically modified by mutation of one or more genes selected from a group consisting of: onipP5, ompP6 and pcp to attenuate the peptidoglycan-binding activity of the encoded protein(s).

7. The hyperblebbing Gram-negative bacterium of claim 2 which is a Moraxella catarrhalis strain which has been genetically modified by down-regulating expression of one or more genes selected from a group consisting of: tolQ, tolR, tolX, tolA and tolB.

8. The hyperblebbing Gram-negative bacterium of claim 2 or 7 which is a Moraxella catarrhalis strain which has been genetically modified by mutation of one or more, genes selected from a group consisting of: ompCD, xompA, pal1, and pal2 to attenuate the peptidoglycan-binding activity of the encoded protein(s).

9. The hyperblebbing Gram-negative bacterium of claims 2-8 which has been further genetically engineered by one or more processes selected from the following group: (a) a process of down-regulating expression of immunodominant variable or non-protective antigens, (b) a process of upregulating expression of protective OMP antigens, (c) a process of down-regulating a gene involved in rendering the lipid A portion of LPS toxic, (d) a process of upregulating a gene involved in rendering the lipid A portion of LPS less toxic, and (e) a process of down-regulating synthesis of an antigen which shares a structural similarity with a human structure and may be capable of inducing an auto-immune response in humans.

10. A preparation of membrane vesicles obtained from the bacterium as defined in any one of claims 1-9.

11. The preparation of membrane vesicles of claim 10 which is capable of being filtered through a 0.22 μm membrane.

12. A sterile, homogeneous preparation of membrane vesicles obtainable by passing the membrane vesicles from the bacterium as defined in any one of claims 1-9 through a 0.22 μm membrane.

13. A vaccine which comprises a bacterium as defined in any one of claims 1-9 or a preparation as defined in any one of claims 10-12 together with a pharmaceutically acceptable diluent or carrier.

14. A vaccine according to claim 13 for use in a method of treatment of the human or animal body.

15. A method of protecting an individual against a bacterial infection which comprises administering to the individual an effective amount of a bacterium as defined in any one of claims 1-9 or a preparation as defined in any one of claims 10-12.

16. A process for preparing a vaccine composition comprising a preparation of membrane vesicles as defined in claims 10-11 which process comprises: (a) inoculating a culture vessel containing a nutrient medium suitable for growth of the bacterium of any one of claims 1-9; (b) culturing said bacterium; (c) recovering membrane vesicles from the medium; and (d) mixing said membrane vesicles with a pharmaceutically acceptable diluent or carrier.

17. The process of claim 16 which further comprises a step after either step (c) or step (d), which step comprises sterile-filtering the preparation of membrane vesicles.

18. A method for producing a hyperblebbing bacterium according to claim 1 which method comprises genetically modifying a Gram-negative bacterial strain by one or more of the following processes: (a) engineering the strain to down-regulate expression of one or more Tol genes; and (b) attenuating the peptidoglycan-binding activity by mutating one or more gene(s) encoding a protein comprising a peptidoglycan-associated site.


SEQUENCE LISTING (98 sequences)

SEQ ID NO: 1 - DNA, 660 bp, Neisseria meningitidis
atgaatttgaaattagtgtttgaatcgggcgatcccgtcctgattggtgtgtttgtgttgatgctgttgatgagtatcgtaacgtggtgtttggttgtcttgcgctgcatcaagctgtatcgggcgcgcaaagggaatgccgccgtcaaacggcatatgcgcgatactttgtcgctgaacgacgcggtcgaaaaagtgcgcgccgtcgatgcgcctttgtccaaactggcgcaagaggcattgcagtcttaccgcaactaccgccgaaacgaagcgtccgaactggcgcaggctttgccgttgaacgagtatttggtcattcaaatccgcaacagtatggcgcagattatgcgccggtttgattacgggatgaccgcgcttgcctccatcggcgcgaccgcgccgtttatcgggctgttcggcacggtttgggggatttaccacgccctgatcaatatcgggcaaagcgggcagatgagtattgcggcggttgccggcccgattggcgaggcactggtggcgacggcggcgggtttgttcgtggcgattccggcggtgttggcatacaacttcctcaatcgcggcacaaaaatactgacccaggatttggatgcgatggcgcacgatttgcacgtccgcctgcttaatcaaaaggatagc

SEQ ID NO: 2 - protein, 220 aa, Neisseria meningitidis
MNLKLVFESGDPVLIGVFVLMLLMSIVTWCLVVLRCIKLYRARKGNAAVKRHMRDTLSLNDAVEKVRAVDAPLSKLAQEALQSYRNYRRNEASELAQALPLNEYLVIQIRNSMAQIMRRFDYGMTALASIGATAPFIGLFGTVWGIYHALINIGQSGQMSIAAVAGPIGEALVATAAGLFVAIPAVLAYNFLNRGTKILTQDLDAMAHDLHVRLLNQKDS

SEQ ID NO: 3 - DNA, 432 bp, Neisseria meningitidis
atggcatttggttcgatgaattccggcgacgattctccgatgtccgacatcaacgttacgccgttggtggacgtgatgctggtgttgctgattgtgtttatgattactatgccggtgctgacgcattccatccctttggaactgccgaccgcgtccgagcagacaaacaagcaggacaaacagcctaaagaccccctgcgcctgacgattgatgcgaacggcggctattatgtcggcggggattctgcaagcaaagtggaaatcggggaagtggaaagccgtctgaaagccgccaaggagcagaatgaaaacgtgattgtggcgattgcggcagacaaggcggtggaatacgattatgtaaacaaagctttagaagccgcccgtcaggcaggaatcaccaaaatcggttttgtaaccgaaaccaaggcgcaa

SEQ ID NO: 4 - protein, 144 aa, Neisseria meningitidis
MAFGSMNSGDDSPMSDINVTPLVDVMLVLLIVFMITMPVLTHSIPLELPTASEQTNKQDKQPKDPLRLTIDANGGYYVGGDSASKVEIGEVESRLKAAKEQNENVIVAIAADKAVEYDYVNKALEAARQAGITKIGFVTETKAQ

SEQ ID NO: 5 - DNA, 1001 bp, Neisseria meningitidis
cataatgattccaacactgaaaaaaccaatcaaacatccaagctgccgcaaaccgctgcggcaaccgcctaattcaattcaaacttgacggggactttaaactccgtccaggcattggcttgaaaatgcccgttttgcgccgccttgcgtgccgcattgtccaaccgggaaaaaccactgcttttcacgattttaacggactcaacatgaccgcccggagaaaccaaaacgctcaaaacaaccgtaccctgctcgtcattctccatagaaagcgtgggataagccgggcgcggaatgctgccgttggcgcgtaaaggattgcctttgctgctgccggctccttccccgtgttcgcctttgacaccgccgctacctttaccgctgccttctccgcgccccgttccgtctcctttggtaccagttcccttatcttccccattgccctgctcgctgtctgctttggcagaagcattgccgggatgttcggcaggtttttcagacggcttctcgaccggtttttccgccggtttcgggacaggcttcgcttccggcttaggctctggtttcggtttttcttcgggtttcggcttttcttcaggtttcggctcttccttaggctgctgaatatccgcatccgcctttttcgtaaccaccggcttcaaaaccggcttgggcggctcgacaggtttgggcggctcgggcacgggttgcggttcgggcgcagcaggcgcgcctgcaccttcgggggcgccgtcccctccgccaaaatcgcccaaatcgacaaattcaataacattgcctgactctatcacgggcagcttgtgcgcctgccagagcaatgccaccattgccaaatgcagcagtgcgacggaaaacacgactgcgggggttaaaattcgttctttatccataattcgggcataataatagcaacaattcctatttgcaacctatttttacaatttttggtcatatgaatgtctgttccgttcacaggcaaa

SEQ ID NO: 6 - DNA, 1003 bp, Neisseria meningitidis
cataatcagctatccttttgattaagcaggcggacgtgcaaatcgtgcgccatcgcatccaaatcctgggtcagtatttttgtgccgcgattgaggaagttgtatgccaacaccgccggaatcgccacgaacaaacccgccgccgtcgccaccagtgcctcgccaatcgggccggcaaccgccgcaatactcatctgcccgctttgcccgatattgatcagggcgtggtaaatcccccaaaccgtgccgaacagcccgataaacggcgcggtcgcgccgatggaggcaagcgcggtcatcccgtaatcaaaccggcgcataatctgcgccatactgttgcggatttgaatgaccaaatactcgttcaacggcaaagcctgcgccagttcggacgcttcgtttcggcggtagttgcggtaagactgcaatgcctcttgcgccagtttggacaaaggcgcatcgacggcgcgcactttttcgaccgcgtcgttcagcgacaaagtatcgcgcatatgccgtttgacggcggcattccctttgcgcgcccgatacagcttgatgcagcgcaagacaaccaaacaccacgttacgatactcatcaacagcatcaacacaaacacaccaatcaggacgggatcgcccgattcaaacactaatttcaaattcataatgattccaacactgaaaaaaccaatcaaacatccaagctgccgcaaaccgctgcggcaaccgcctaattcaattcaaacttgacggggactttaaactccgtccaggcattggcttgaaaatgcccgttttgcgccgccttgcgtgccgcattgtccaaccgggaaaaaccactgcttttcacgattttaacggactcaacatgaccgcccggagaaaccaaaacgctcaaaacaaccgtaccctgctcgtcattctccatagaaagcgtgggataagccgggcgcggaatgctgccgttggcgcgtaaaggattgcctttgctgctgccggc

SEQ ID NO: 7 - DNA, 729 bp, Neisseria meningitidis
atgaccaaacagctgaaattaagcgcattattcgttgcattgctcgcttccggcactgctgttgcgggcgaggcgtccgttcagggttacaccgtaagcggccagtcgaacgaaatcgtacgcaacaactatggcgaatgctggaaaaacgcctactttgataaagcaagccaaggtcgcgtagaatgcggcgatgcggttgctgcccccgaacccgagccagaacccgaacccgcacccgcgcctgtcgtcgttgtggagcaggctccgcaatatgttgatgaaaccatttccctgtctgccaaaaccctgttcggtttcgataaggattcattgcgcgccgaagctcaagacaacctgaaagtattggcgcaacgcctgagtcgaaccaatgtccaatctgtccgcgtcgaaggccataccgactttatgggttctgacaaatacaatcaggccctgtccgaacgccgcgcatacgtagtggcaaacaacctggtcagcaacggcgtacctgtttctagaatttctgctgtcggcttgggcgaatctcaagcgcaaatgactcaagtttgtgaagccgaagttgccaaactgggtgcgaaagtctctaaagccaaaaaacgtgaggctctgattgcatgtatcgaacctgaccgccgtgtggatgtgaaaatccgcagcatcgtaacccgtcaggttgtgccggcacacaatcatcaccaacactaa

SEQ ID NO: 8 - protein, 242 aa, Neisseria meningitidis
MTKQLKLSALFVALLASGTAVAGEASVQGYTVSGQSNEIVRNNYGECWKNAYFDKASQGRVECGDAVAAPEPEPEPEPAPAPVVVVEQAPQYVDETISLSAKTLFGFDKDSLRAEAQDNLKVLAQRLSRTNVQSVRVEGHTDFMGSDKYNQALSERRAYVVANNLVSNGVPVSRISAVGLGESQAQMTQVCEAEVAKLGAKVSKAKKREALIACIEPDRRVDVKIRSIVTRQVVPAHNHHQH

SEQ ID NO: 9 - DNA, 729 bp, Neisseria meningitidis
atgaccaaacagctgaaattaagcgcattattcgttgcattgctcgcttccggcactgctgttgcgggcgaggcgtccgttcagggttacaccgtaagcggccagtcgaacgaaattgtacgcaacaactatggcgaatgctggaaaaacgcctactttgataaagcaagccaaggtcgcgtagaatgcggcgatgcggttgctgcccccgaacccgagccagaacccgaacccgcacccgcgcctgtcgtcgttgtggagcaggctccgcaatatgttgatgaaaccatttccctgtctgccaaaaccctgttcggtttcgataaggattcattgcgcgccgaagctcaagacaacctgaaagtattggcgcaacgcctgggtcaaaccaatatccaatctgtccgcgtcgaaggccataccgactttatgggttctgacaaatacaatcaggccctgtccgaacgccgcgcatacgtagtggcaaacaacctggtcagcaacggcgtacctgtttctagaatttctgctgtcggcttgggcgaatctcaagcgcaaatgactcaagtttgtgaagccgaagttgccaaactgggtgcgaaagtctctaaagccaaaaaacgtgaggctctgattgcatgtatcgaacctgaccgccgcgtggatgtgaaaatccgcagcatcgtaacccgtcaggttgtgccggcacacaatcatcaccaacactaa

SEQ ID NO: 10 - DNA, 1007 bp, Neisseria meningitidis
aaaatgcccgcgcgatgctgctgcccgcattgaatgcaaattcataagtaatcagcggaaacctcgccaaatcttcaatacggagggggtttctgcattcgagcaaggggtggtcgttcggtacgataaccgcatgagtccagtcatagcagggaagttttcccagttcgggatggtcgtctatccgttccgtaacaatcgccaagtccgcctcgcctgaggtaaccatacgtgcgatggcggcagggctcccctgtttgatggtcaggttgactttcggatagcgtttcacaaaatcggcaacaatcaagggtagggcatagcgtgcctgagtatgcgtcgtggcaaccgtcagcgaaccgctgtcctgtccggtaaactcgctgccgatatttttaatgttctgaacatcgcgcaaaatacgttccgcaatatccaaaaccaccttgcccggctgcgagaccgaaaccacgcgcttgccgctgcggataaaaatctgaatgccgatttcttcttccagcaatttgatttgtttggagatgccgggttgcgaagtaaacaaggcttcggccgcttcggaaacgttcaggttgtgctggtaaacttctaaggcgtatttcaattgttgtaatttcatggcgggtcggtgtgggtctgtgtcgggtggctgaacattgtttataatttatcatattttcttgccggtacggtatggggctttgccgttgtgtttgttgtttttgtgcaacggcaatcgtgcgatatggaaaaaatccccctaaagtaatgacacggaattgatttttcggcatgatagactatcaggaaacaggctgttttacggttgttttcaggcgttgagtattgacagtccgccccctgcttctttatagtggagactgaaatatccgatttgccgccatgtttctacagcggcctgtatgttggcaattcagcagttgcttctgtatctgctgtacaaatttaatgagggaataaaatgaatg

SEQ ID NO: 11 - DNA, 687 bp, Haemophilus influenzae
ttagtgaggggctttaccaaaggcttgacggtgtaaaatcgtcgtaaattcatcaataaaattaccgtaatcttgttcaatggcattcactcgtaagcttaaacggttataagccattactgcaggaattgcggcaaataaaccaatcgcagtggcaatcaaggcctcagcgatacctggcgctaccatctgtaacgttgcttgttttgcaccacttaatgccataaaagcgtgcatgataccccaaacagtgccgaataaaccaatataagggctaacagatgccactgtggctaaaaatggaactcggttttccaaactttcaatctcacggttcatcgcaagattcatcgcgcgcattgtgcctttaataatcgcttcaggtgcatctggatttacttgttttaaacgtgaaaattctttaaatcccacgcaaaaaatttgttcgctgcccgttaatccatcgcgacgattagatagcccttcataaagtttatttaaatcttctcctgaccagaaacgatcttcaaacgtacgcgcttcttttaaggcattcgttaaaatacgactacgttgaatgataattgcccaagatatgattgagaaagaaatcaaaatcacaattaccagttgcacaacaatacttgcttttagaaaaagatctaaaaaattcaattctgcagtcat

SEQ ID NO: 12 - protein, 228 aa, Haemophilus influenzae
MTAELNFLDLFLKASIVVQLVIVILISFSIISWAIIIQRSRILTNALKEARTFEDRFWSGEDLNKLYEGLSNRRDGLTGSEQIFCVGFKEFSRLKQVNPDAPEAIIKGTMRAMNLAMNREIESLENRVPFLATVASVSPYIGLFGTVWGIMHAFMALSGAKQATLQMVAPGIAEALIATAIGLFAAIPAVMAYNRLSLRVNAIEQDYGNFIDEFTTILHRQAFGKAPH

SEQ ID NO: 13 - DNA, 420 bp, Haemophilus influenzae
ctaaatgggatttgtcattaaacctacagatttaatgcctgcaagatgaagtaaattcaatgccttaatcacttcttcataaggtacttctttagctccgcctactaaaaatagcgtattattatccttatcaaattcctgtctagataattgagtaaccatttcttctgttaaaccttcttgacgttctccgccaatagaaatcgcatattttccaatgcctgccacttcaagaatgacgggtactttatcttcattagaaacctcttggctttgcacagaatcaggcaattcaacttgaacgctttgactaataataggggcggttgccataaaaattaacactaaaactaaaagcacatctaaaaaaggcacaatattaatttcagatttaattgctttacgctgacgacgagccat

SEQ ID NO: 14 - protein, 139 aa, Haemophilus influenzae
MARRQRKAIKSEINIVPFLDVLLVLVLIFMATAPIISQSVQVELPDSVQSQEVSNEDKVPVILEVAGIGKYAISIGGERQEGLTEEMVTQLSRQEFDKDNNTLFLVGGAKEVPYEEVIKALNLLHLAGIKSVGLMTNPI

SEQ ID NO: 15 - DNA, 1119 bp, Haemophilus influenzae
ttatcgaatatcaaagtcaataattggtgatttatatttttcataaatttcatctgatggcgcagctggaacttttttcgttctagccaccgcacttaatgcagctgaacaaatatcatcagagcctgaaattttttgataccccaagattgtgccatctcgacctaattgaattttaatacgacaaacctttcctgcaaaatttggatcttttaagaaacgacgttgaatctctttcttaattacacctgcgtattgatccccaaccttaccaccatcgccagagccaagtgcagcaccgctaccttgagttccacctttatttgtgtttccccctttagatgcactaccgccaccaatatctccgccatttaagaaatcatctaggcttgcttgatctgctttacgtttcgcttccgtagcagctttagcttctgcatcagcttttgcttttgcctctgctgccgctttcgcttttgcctccgcttcagcctttgctttagcttcagcaacggcttttgccttagcttcggcttctagtttcgccttagcttccgcctcttgttttgctttttgagcagcaatttctgctgctttcgctttagcctcttcttcagcttgttttgccgcggcagctaaacgtttagcctctgcatctgcttttaattttgcagcttcagccgcttgtttagccttagcctcttcagcttgcttctgtttttccaacgcttcttgacgagcttgctcttgttgtttttttatttcttgctgacgttgctgttcttgctgacgttttaactcttcttgtcgttgaacttcttgttgatgcttaatctcttcttgattaggctcaggtggtttttcttccacaacaggttctgggcgtttttgtttatccgcttgcccttttttttgttgttgaatacgcccccattcctgagcagccgtaccagtatcaacaatcactgcccctattacatctccttcaccttctccaccacccataatttcaacagtgtgataaagtgagcttaaaatcaataagccaaacaagataaagtgcaaaaggatagaaatagcaaaagcattgattcctttcttttgtcgattattttgcac

SEQ ID NO: 16 - protein, 372 aa, Haemophilus influenzae
MQNNRQKKGINAFAISILLHFILFGLLILSSLYHTVEIMGGGEGEGDVIGAVIVDTGTAAQEWGRIQQQKKGQADKQKRPEPVVEEKPPEPNQEEIKHQQEVQRQEELKRQQEQQRQQEIKKQQEQARQEALEKQKQAEEAKAKQAAEAAKLKADAEAKRLAAAAKQAEEEAKAKAAEIAAQKAKQEAEAKAKLEAEAKAKAVAEAKAKAEAEAKAKAAAEAKAKADAEAKAATEAKRKADQASLDDFLNGGDIGGGSASKGGNTNKGGTQGSGAALGSGDGGKVGDQYAGVIKKEIQRRFLKDPNFAGKVCRIKIQLGRDGTILGYQKISGSDDICSAALSAVARTKKVPAAPSDEIYEKYKSPIIDFDIR

SEQ ID NO: 17 - DNA, 1284 bp, Haemophilus influenzae
ttatttagttaagtatggagaccaagctggaaatttaacttgaccatcacttcctggaaggctcgccttaaagcgaccatctgcggaaaccaattgtagcacctttcctaagccctgtgtagaactataaataatcataattccatttggagagaggcttgggctttcgcctagaaaagatgtactaagtacctctgaaacgcccgttgtgagatcttgtttaactacattattgttaccattaatcatcacaagtgtttttccatctgcactaatttgtgcgctaccgcgaccacccactgctgttgcactaccaccgcttgcatccattcgataaacttgtggcgaaccacttctatcggatgtaaataaaattgaatttccgtctggcgaccacgctggttcagtattattacccgcaccactcgtcaattgagtaggtgtaccgccatttgctcccataacgtaaatattcagaacaccatcacgagaagaagcaaaagctaaacgagaaccatctggcgaaaaggctggtgcgccattatgcccttgaaaagatgccactactttacgtgcgccagaatttaaatcctgtacaacaagttgtgattttttattttcaaacgatacataagccaaacgctggccgtctggagaccaagctggagacataattggttgggcactacgattgacgataaattgattatagccatcataatctgctacacgaacttcataaggttgcgaaccgccatttttttgcacaacataagcgatacgagttctaaaggcaccacggatcgcagttaatttttcaaaaacttcatcgctcacagtatgcgcgccatagcgtaaccatttatttgttactgtatagctattttgcattaatacagtccctggcgtacctgatgcaccaaccgtatcaattaattgataagtaatactataaccattacccgatggaaccacttgcccaattacaattgcgtcaattccaatattcgaccaagcctcaggatttacctctgcagctgaagttgggcgttgaggcatttgagaaaccgcaataggattaaacttaccactgttacgtaaatcatctgcaacaattttactaatatcttctggtgcagaaccaacaaatggcacgacagcaataggacgcgcaccatcaaccccttcatcaatgacaatgcgtacttcatcgccagcgaatgcattgcttccaacagcaagtacaatcgcgaatacgctcactaaacgttttaataatttcat

SEQ ID NO: 18 - protein, 427 aa, Haemophilus influenzae
MKLLKRLVSVFAIVLAVGSNAFAGDEVRIVIDEGVDGARPIAVVPFVGSAPEDISKIVADDLRNSGKFNPIAVSQMPQRPTSAAEVNPEAWSNIGIDAIVIGQVVPSGNGYSITYQLIDTVGASGTPGTVLMQNSYTVTNKWLRYGAHTVSDEVFEKLTAIRGAFRTRIAYVVQKNGGSQPYEVRVADYDGYNQFIVNRSAQPIMSPAWSPDGQRLAYVSFENKKSQLVVQDLNSGARKVVASFQGHNGAPAFSPDGSRLAFASSRDGVLNIYVMGANGGTPTQLTSGAGNNTEPAWSPDGNSILFTSDRSGSPQVYRMDASGGSATAVGGRGSAQISADGKTLVMINGNNNVVKQDLTTGVSEVLSTSFLGESPSLSPNGIMIIYSSTQGLGKVLQLVSADGRFKASLPGSDGQVKFPAWSPYLTK

SEQ ID NO: 19 - DNA, 970 bp, Haemophilus influenzae
tcattgcatactccgaaaaattattttaagtgatgaaacgccgctttaacttctttgggaaacgccactggtttcatcttgcctagatcaacacaggctaccttaacagtagcctttgataacatcagggtgttgcgcatcagtctctgttcaaaaaggattgtagccccttttacttctgaaacctctgtttccaccataagtaaatcatccaattttgctgccacgcaataatcaatggcgagcgttttgacaacaaatgcgagttgttgttcctctagtaaggtttgttgcgtaaaatttaatgtacgcaaatattctgttcttgctcgttcaaaaaaatgcaaatagcgagcgtgatacactacgccacctgcatcagtatcttcataatacacacgaacaggaaaagaaaagccattatccaacatattctcacccaattggtcgcaataaaccgtgtattctagaaccagtttttgggataagcaagctatctatgaaaaactcaataagattttattcattttaaaacatctaaaatttttaccgcacttttagcctgactagcaaaagataaggtaatgacaaatcatttttaacctttctcattgagtaaaatctattcaaaacataaccgttctttaaaaatagcctctatgtaatcttaagccaccagtatttttattcttgatatttagcgtttctatgcgacaatctttgcggttatttactttaaaaatatgttttactagatggattacgaaaatcaaattgccaatattttctcactaaatggcgaattaagccaaaatatcaaaggttttcgtcctcgagctgaacaacttgaaatggcatatgctgtaggtaaagcaattcaaaataaatcttcccttgttattgaagctggaacgggtacaggaaaaacctttgcatatctcgcacctgctttagtttttggtaaaaaaac

SEQ ID NO: 20 - DNA, 1059 bp, Haemophilus influenzae
  misc_feature (1)...(1059): n = A,T,C or G
atgaaaaaaactgcaatcgcattagtagttgctggtttagcagcagcttcagtagctcaagcagctccacaagaaaacactttctacgctggcgttaaagctggtcaagcatcttttcacgatggacttcgtgctctagctcgtgaaaagaatgttggttatcaccgtaattctttcacttatggtgtattcggtggttatcaaattttaaatcaaaataacttaggtttagcggttgaattaggttacgacgatttcggtcgtgccaaaggtcgtgaaaaaggtagaactgttgctaaacacactaaccacggtgcgcatttaagcttanaaggtagctatgaagtgttagaaggtttagatgtttatggtaaagcaggtgttgctttagttcgttctgactataaattgtacaataaaaatagtagtactcttaaagacctaggcgaacatcacagagcacgtgcctctggtttatttgcagtaggtgcagaatatgcagtattaccagaattagcagttcgtttagaataccaatggctaactcgcgtaggtaaataccgccctcaagataaaccaaataccgcaattaactacaacccttggattggttctatcaacgcaggtatttcttaccgctttggtcaaggcgaagcaccagttgttgcagcacctgaaatggtaagcaaaactttcagcttaaattctgatgtaacttttgcatttggtaaagcaaacttaaaacctcaagcgcaagcaacattagacagcgtctatggcgaaatttcacaagttaaaagtgcaaaagtagcggttgctggttacactgaccgtattggttctgacgcgttcaacgtaaaactttctcaagaacgtgcagattcagtagctaactactttgttgctaaaggtgttgctgcagacgcaatctctgcaactggttacggtgaagcaaacccagtaactggcgcaacttgtgaccaagttaaaggtcgtaaagcacttatcgcttgtcttgctccagaccgtcgtgtagaaatcgcggtaaacggtactaaa

SEQ ID NO: 21 - protein, 353 aa, Haemophilus influenzae
  VARIANT (1)...(353): Xaa = Any Amino Acid
MKKTAIALVVAGLAAASVAQAAPQENTFYAGVKAGQASFHDGLRALAREKNVGYHRNSFTYGVFGGYQILNQNNLGLAVELGYDDFGRAKGREKGRTVAKHTNHGAHLSLXGSYEVLEGLDVYGKAGVALVRSDYKLYNKNSSTLKDLGEHHRARASGLFAVGAEYAVLPELAVRLEYQWLTRVGKYRPQDKPNTAINYNPWIGSINAGISYRFGQGEAPVVAAPEMVSKTFSLNSDVTFAFGKANLKPQAQATLDSVYGEISQVKSAKVAVAGYTDRIGSDAFNVKLSQERADSVANYFVAKGVAADAISATGYGEANPVTGATCDQVKGRKALIACLAPDRRVEIAVNGTK

SEQ ID NO: 22 - DNA, 459 bp, Haemophilus influenzae
atgaacaaatttgttaaatcattattagttgcaggttctgtagctgcattagcagcttgtagttcatctaacaacgatgctgcaggcaatggtgctgctcaaacttttggcggttactctgttgctgatcttcaacaacgttacaataccgtttatttcggttttgataaatatgacattactggtgaatacgttcaaatcttagacgcgcacgctgcatatttaaatgcaacgccagctgctaaagtattagtagaaggtaacactgatgaacgtggtacaccagaatacaacatcgcattaggccaacgtcgtgcagatgcagttaaaggttatttagctggtaaaggtgttgatgctggtaaattaggcacagtatcttacggtgaagaaaaacctgcagtattaggtcatgatgaagctgcatattctaaaaaccgtcgtgcagtgttagcgtac

SEQ ID NO: 23 - protein, 153 aa, Haemophilus influenzae
MNKFVKSLLVAGSVAALAACSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAY

SEQ ID NO: 24 - DNA, 462 bp, Haemophilus influenzae
atgaacaaatttgttaaatcattattagttgcaggttctgtagctgcattagcggcttgtagttcctctaacaacgatgctgcaggcaatggtgctgctcaaacttttggcggatactctgttgctgatcttcaacaacgttacaacaccgtatattttggttttgataaatacgacatcaccggtgaatacgttcaaatcttagatgcgcacgcagcatatttaaatgcaacgccagctgctaaagtattagtagaaggtaatactgatgaacgtggtacaccagaatacaacatcgcattaggacaacgtcgtgcagatgcagttaaaggttatttagcaggtaaaggtgttgatgctggtaaattaggcacagtatcttacggtgaagaaaaacctgcagtattaggtcacgatgaagctgcatattctaaaaaccgtcgtgcagtgttagcgtactaa

SEQ ID NO: 25 - protein, 153 aa, Haemophilus influenzae; identical to SEQ ID NO: 23

SEQ ID NO: 26 - DNA, 465 bp, Haemophilus influenzae
atgaaaaaaacaaatatggcattagcactgttagttgcttttagtgtaactggttgtgcaaatactgatattttcagcggtgatgtttatagcgcatctcaagcaaaggaagcgcgttcaattacttatggtacgattgtttctgtacgccctgttaaaattcaagctgataatcaaggtgtagttggtacgcttggtggtggagctttaggtggtattgctggtagtacaattggcggtggtcgtggtcaagctattgcagcagtagttggtgcaattggcggtgcaatagctggaagtaaaatcgaagaaaaaatgagtcaagtaaacggtgctgaacttgtaattaagaaagatgatggtcaagagatcgttgttgttcaaaaggctgacagcagtttttgtagcttggtcgccgagttcgtatttgttggtggcggctcaagcttaaatgtttctgtgcta

SEQ ID NO: 27 - protein, 155 aa, Haemophilus influenzae
MKKTNMALALLVAFSVTGCANTDIFSGDVYSASQAKEARSITYGTIVSVRPVKIQADNQGVVGTLGGGALGGIAGSTIGGGRGQAIAAVVGAIGGAIAGSKIEEKMSQVNGAELVIKKDDGQEIVVVQKADSSFCSLVAEFVFVGGGSSLNVSVL

SEQ ID NO: 28 - DNA, 690 bp, Moraxella catarrhalis
atgaacgaatccattagcctaatctcgctggtcattgaagcaagcgttgttgttaaattggtcatggcgatactgcttttgctgtctacaatcagttgggtactgatttttcatctgggtaccaaaattggcggtattgccaagtttgataagcgatttgagcgatggttttggactgatgatatcgatcatcagctgtctgttgtgcaagcagaatcagagcgtgcagggcttgagctgattttttatacaggtttttatgatcaaaatcaccaagaccaagattcttcactaagtgatgataaaaaagtgcaaatcgttgagcgtcgcttgcgtatggcattaggcagtgagcaggtgcatcttgaaaaaggattatcaacgcttgcaacgattggttctgtttcaccttatatcggactatttggtacagtatggggcattatgaatgcatttattggcttgggtcaagccgaatcggttggtcttgcaaccgttgcaccgagcattgctgaggcattgattgcaacagcacttggtttatttgcggccattcctgcgacgatggcatataatcactttgccaccaaatccaatacactgtatgaaaatcgtagcctattttgtgaaggcttaataagtgcattggtgacaaatctggcaaaaaagaacaccgcatcaactttatag

SEQ ID NO: 29 - protein, 229 aa, Moraxella catarrhalis
MNESISLISLVIEASVVVKLVMAILLLLSTISWVLIFHLGTKIGGIAKFDKRFERWFWTDDIDHQLSVVQAESERAGLELIFYTGFYDQNHQDQDSSLSDDKKVQIVERRLRMALGSEQVHLEKGLSTLATIGSVSPYIGLFGTVWGIMNAFIGLGQAESVGLATVAPSIAEALIATALGLFAAIPATMAYNHFATKSNTLYENRSLFCEGLISALVTNLAKKNTASTL

SEQ ID NO: 30 - DNA, 435 bp, Moraxella catarrhalis
atggtaacttccaatcgattcgctcgtcgccaaagaccgctaaatagtgacatgaatgttgtgccttacattgatgtgatgttggtgcttttggtgatatttatcgtaacagcaccaatgcttgctacaggtattgaggtatcactgccaaaagagcagaccaaacccatcacacaagctgacaagctgcctgtcattgtcagcattcaggcagatggcaatctgtatgtcagccataaaaatgccatcgatgtgccaatcacgcctgacaagctagataccctgctacgccagatgcaccaagacaataccgatttacaagtgatggtcaatgccgatgcagataatgcctacagccgaattatgcagattatggcattgattcaaaatgttggtatcacccaagtgagtttgcttagcgaatctgttcaataa

SEQ ID NO: 31 - protein, 144 aa, Moraxella catarrhalis
MVTSNRFARRQRPLNSDMNVVPYIDVMLVLLVIFIVTAPMLATGIEVSLPKEQTKPITQADKLPVIVSIQADGNLYVSHKNAIDVPITPDKLDTLLRQMHQDNTDLQVMVNADADNAYSRIMQIMALIQNVGITQVSLLSESVQ

SEQ ID NO: 32 - DNA, 828 bp, Moraxella catarrhalis
atgataattcataaggcaaatcaatcgatgcgtttatccgataatcatccaacagtcaattttgataaatctgcgctaattttaccaattttagccagtgttttattacataccgtcatcatcatagcggtagcagcaccactgattacaccgcctactaagcctaatactactattcagaccgctttggtaggtcaagaggcttttaatcgtgccaagacggccttgagcaatcatcatgccaatcaaaacaagccaactgccaccaacacttcaagtaccatcactgccaatgataatgataatgcatttatgcaagctcaaaatcagcatcgttatcacccacaggtttctacttctgccaccacgacccaagcgtatcatccaccacccaactcagcaccctttgaatcaaattcaccaaatatacaaaatcaaccaacaaacgctcacgccaagctggctgaatattctaatcatgtctcagaccttgagcagtcaaatcataccgagtctacgccaagccgagcacaaatcaatgccgccatcacctcggtcaaacatcgtattgaagccatttggcaacgctatcctaagcagcccaatcaaaccatcacctttcaggttaatatgaatcaacaaggcgatgtgacctcaatccaattcggtggtggccatcctgatttgcgtgaatctgtagaagcggcggtatatgctgccgcaccattttatgaacttggcggtatgcgtgacagtatccgcctgcagttcaccacagagcagctaattatggataataaccaaacaaccaatgagcctaatcactaa

SEQ ID NO: 33 - protein, 275 aa, Moraxella catarrhalis
MIIHKANQSMRLSDNHPTVNFDKSALILPILASVLLHTVIIIAVAAPLITPPTKPNTTIQTALVGQEAFNRAKTALSNHHANQNKPTATNTSSTITANDNDNAFMQAQNQHRYHPQVSTSATTTQAYHPPPNSAPFESNSPNIQNQPTNAHAKLAEYSNHVSDLEQSNHTESTPSRAQINAAITSVKHRIEAIWQRYPKQPNQTITFQVNMNQQGDVTSIQFGGGHPDLRESVEAAVYAAAPFYELGGMRDSIRLQFTTEQLIMDNNQTTNEPNH

SEQ ID NO: 34 - DNA, 1263 bp, Moraxella catarrhalis
atgaaatcacccattaccaaagtttgccttgctctgaccataagcttttctgccgctttgacgcacacttatgctgatgatgaattgattgtgattagcgaacaagttgctccgagtcaataccccgtggcagtcatgcctttttcagaagctcatcaaatgagtcattatctaagcctggcaggtcttggtactactcaccaaaacctgccacagcacactcagacgaatagcgacattctgaataatctgaccgcatggcgtaaccgaggatttgaatatattattttggcacagtcgcatcaaattttgggaaataagcttgcaattaactatgaaattattgatactgccaatggtttggtaagcgtcaagcatacccaaattagcgataaccaccctgcttctatccaagctgcctatcgtcaaatcagcgatacaatctatcaaatcatcacaggccagccatcagatttgatgggtaaaatcgcctatgtggaagaaagcggatcgccacaaaataaaatctcatctcttaaattgattgatccaagcggtcagcttatccgtacgctagataccgtcaatggatcaattataacgccgacattttcccccgatggcttgagtattgcttatagtgtacaaacaaaaaataatctgcccatcatttatattgtgtctgtatcaggtggcacaccaaagctcgtcacgccattttggggtcataatttggcaccaagtttttcaccagatggtagcagtatcttattttcaggtagccacgagaataataacccgaacatttatcgtcttaatttacataccaatcacttagatacgctcactacattcaacggtgctgagaatgcaccaaattatttggcagatgcgtcaggatttatttatactgctgataaaggtacacgccgccaaagcctatatcgctatgattttggcacgacgcatagcacccaaatcgcctcttatgccaccaatccacgcttaagcccagatggatcaaagcttgtatatttatcaggtggacaaatcatcatcgccaataccaaaggccgtatccaacaaagttttagggtgttaggcactgatgtatcagccagcttttcaccatcaggcacacggattatatatacatccaaccaaggcaataaaaaccagctgatgatccgttcgctatcaagtaatgccatacgcaccatcccaacatcaggcacggtgcgtgatccgatttggtcaaaataa

SEQ ID NO: 35 - protein, 420 aa, Moraxella catarrhalis
MKSPITKVCLALTISFSAALTHTYADDELIVISEQVAPSQYPVAVMPFSEAHQMSHYLSLAGLGTTHQNLPQHTQTNSDILNNLTAWRNRGFEYIILAQSHQILGNKLAINYEIIDTANGLVSVKHTQISDNHPASIQAAYRQISDTIYQIITGQPSDLMGKIAYVEESGSPQNKISSLKLIDPSGQLIRTLDTVNGSIITPTFSPDGLSIAYSVQTKNNLPIIYIVSVSGGTPKLVTPFWGHNLAPSFSPDGSSILFSGSHENNNPNIYRLNLHTNHLDTLTTFNGAENAPNYLADASGFIYTADKGTRRQSLYRYDFGTTHSTQIASYATNPRLSPDGSKLVYLSGGQIIIANTKGRIQQSFRVLGTDVSASFSPSGTRIIYTSNQGNKNQLMIRSLSSNAIRTIPTSGTVRDPIWSK

SEQ ID NO: 36 - DNA, 2450 bp, Moraxella catarrhalis
ggcgactggcggattgtggagtatcgctgtactgtgtactcattgcacccatggcatcaaacatacacgattgcgtccaatgctcactttcaccgccgcctgccagtacgatatcagccttaccaagttgaatcagctccatggcatgaccgacacagtggcttgaagtggcacaggcagaagatagcgagtaagacaagcccttgatttttagccccgtcgctaaggccgcggataccgagcttgccatgattttgggaactgccattgcacctacgccacgcaagcctttttcacgcatggcatccgcagctgccaccacatccgcagtagaagcaccgcccgatgctgcaaccaccgaaaccctaggattgtcagtgatggtgtcaatgctaagccctgcgtttttgattgctgataaagcactgatatatgcataaaggctggcgttgctcataaagcgctttaatttacgatcaatgcctgtggtgtccaagtcatcatgatctatactacctgccacgcatgatttaaatcccaaatcggcatattcttgcttaaagcgaatgcctgaacgcccattttctaaggcctccttgacggtatctaaatcattacccaagcaagaaacaatgcctgcacctgtgatgacaactcgtttcataatttcatcctaaaaagtttacagttgtaatcttgctattgtaacaaattattccaacacttagggaaattttcccaaaattttcataaaaataggtgaaaatgactaaagatagacaagggtttaccaaatatttagttattcatcaattggcgacggtatttatgaacatttaataacatttatgttgtatattatcactaggcgtagtttagtttttgtgataatctttagaagataatttttatgacaatttcatacaattaatgaggttggacatacgatagataaaagtaaattgactttttgtattttatgtcaaaacctgaatcttaataccaaaatcatggagtaactgatgacaaaatcaactcaaaaaaccaccaaacaaacacaacacagccatgatgatcaagtcaaagagctggctcaagaagtcgctgaatatgatgatgttgaaattgttgctgaagtagatatcgacaatcaagctgtctctgatgttttgattattcgtgatacggataccaaagctgaccaagcagatcacactgatgacgcatctaaagcagatgatgagactgtggtagatggcgttaaacaaaaagctcaagaggctaaagaagattttgaaaataaagcacaagatcttcaagataaagctactgagaagcttgaagtcgccaaagaagctacccaagacaaggtagagaaaactcaaagtttagttgaggatatcaaggataaagcccaatctttgcaagaagatgctgccgatacagttgaagcgttaaaacaagcggccagtgataaggttgagactaccaaagctgaagctcaatcactaaaagatgatgctactcaaacatttgaatcagccaaacaagcggttgaaggcaaagtagaagccatcaaagagcaagtcttagatcaggttgactccctaaaagacgataccgatcaagataatactgatcaagatcaagaaaaacagaccctaaaagataaggcggtgcaagctgccaccgctgctaaacgcaaagttgaagatgtggtagatgatgtcaaacacaccaccgaatctttcaaaaataccgcaagcgaaaaaatagatgagattaagcaagctgctgttgacaaaacagaagaggtcaaatctcagcttagccaaaaagctgatgccctaaaatcttctggcgaagaactcaagcaaacagctcaaacggctgctaatgatgccattacagaggctcaagctgccgtagtaagtggttcggttgctgccgctgattcggcacaatcaaccgctcaaagtgcaaaagataagctcaatcagctctttgaacaaggtaagtccgctttggatgaaaaagttcaagaattgggcgagtaatatggtgcaactgagaaaattaatgcagtcagcgaatatgtagatctggctacccaagtcattaaagaagaagcacaagcactacaaaccaatgcccaagaatctctacaagctgccaaagcggctggcgaagagtatgacgctacccacgaagataagggtttgaccactaaacttggtacagtgggtgcctatttgtctggcatgtatggcattagccaaaataaaaataaccattaccaaggcgttgacttgcatcgtgaaagttttgataaagatgcatttcatgcccaaagcagtttttttgcagggacaaatatttggtgccaaagcagttgcagctaagaatgtggcagctaaagttgttcctcaatctaaatttgaagccatcgg

SEQ ID NO: 37 - protein, 458 aa, Moraxella catarrhalis
MTKSTQKTTKQTQHSHDDQVKELAQEVAEYDDVEIVAEVDIDNQAVSDVLIIRDTDTKADQADHTDDASKADDETVVDGVKQKAQEAKEDFENKAQDLQDKATEKLEVAKEATQDKVEKTQSLVEDIKDKAQSLQEDAADTVEALKQAASDKVETTKAEAQSLKDDATQTFESAKQAVEGKVEAIKEQVLDQVDSLKDDTDQDNTDQDQEKQTLKDKAVQAATAAKRKVEDVVDDVKHTTESFKNTASEKIDEIKQAAVDKTEEVKSQLSQKADALKSSGEELKQTAQTAANDAITEAQAAVVSGSVAAADSAQSTAQSAKDKLNQLFEQGKSALDEKVQELGEYGATEKINAVSEYVDLATQVIKEEAQALQTNAQESLQAAKAAGEEYDATHEDKGLTTKLGTVGAYLSGMYGISQNKNNHYQGVDLHRESFDKDAFHAQSSFFAGTNIWCQSSCS

SEQ ID NO: 38 - DNA, 1362 bp, Moraxella catarrhalis
atgaaatttaataaaatcgctcttgcggtcatcgcagccgttgcagctccagttgcagctccagttgctgctcaagctggtgtgacagtcagcccactactacttggctatcattacactgacgaagcccacaatgatcaacgcaaaatcttacgcactggcaagaagctagagctagatgctactaatgcacctgcaccagctaatggcggtgtcgcactggacagtgagctatggactggtgctgcgattggtatcgaacttacgccatcaactcagttccaagttgaatatggtatctctaaccgtgatgcaaaatcttcagacaaatctgcacatcgctttgatgctgagcaagaaaccatcagcggtaactttttgattggtactgagcagttcagcggctacaatccaacaaataaattcaagccctatgtcttggttggtgcaggtcaatctaaaattaaagtaaatgcaattgatggttatacagcagaagtagccaatgggcaaaacattgcaaaagatcaagctgtaaaagcaggtcaagaagttgctgagtctaaagacaccatcggtaacctaggtcttggtgctcgctacttagtcaatgatgcccttgcacttcgtggtgaagcccgtgctatccataattttgataacaaatggtgggaaggcttggcgttggctggtttagaggtaactttgggtggtcgtttggcacctgcagtaccagtagcaccagtggcagaacctgttgctgaaccagttgttgctccagcacctgtgatccttcctaaaccagaacctgagcctgtcattgaggaagcaccagctgtaattgaagatattgttgttgattcagacggagatggtgtgcctgatcatctggatgcctgcccaggaactccagtaaacactgttgttgatccacgcggttgcccagtacaggttaatttggtagaagagcttcgccaagagttgcgtgtattctttgattatgataaatcaatcatcaaaccacaataccgtgaagaagttgctaaggttgctgcgcaaatgcgtgaattcccaaatgcaactgcaaccattgaaggtcacgcatcacgcgattcagcacgctcaagtgcacgctacaaccagcgtctatctgaagctcgtgctaatgctgttaaatcaatgctatctaacgaatttggtatcgctccaaaccgcctaaatgcagttggttatggctttgatcgtcctatcgctccaaatactactgctgaaggtaaagcgatgaaccgtcgtgtagaagcagtaatcactggtagcaaaacaacgactgttgatcaaaccaaagatatgattgttcaataa

SEQ ID NO: 39 - protein, 453 aa, Moraxella catarrhalis
MKFNKIALAVIAAVAAPVAAPVAAQAGVTVSPLLLGYHYTDEAHNDQRKILRTGKKLELDATNAPAPANGGVALDSELWTGAAIGIELTPSTQFQVEYGISNRDAKSSDKSAHRFDAEQETISGNFLIGTEQFSGYNPTNKFKPYVLVGAGQSKIKVNAIDGYTAEVANGQNIAKDQAVKAGQEVAESKDTIGNLGLGARYLVNDALALRGEARAIHNFDNKWWEGLALAGLEVTLGGRLAPAVPVAPVAEPVAEPVVAPAPVILPKPEPEPVIEEAPAVIEDIVVDSDGDGVPDHLDACPGTPVNTVVDPRGCPVQVNLVEELRQELRVFFDYDKSIIKPQYREEVAKVAAQMREFPNATATIEGHASRDSARSSARYNQRLSEARANAVKSMLSNEFGIAPNRLNAVGYGFDRPIAPNTTAEGKAMNRRVEAVITGSKTTTVDQTKDMIVQ

SEQ ID NO: 40 - DNA, 2461 bp, Moraxella catarrhalis
atgtgtttgcattgattgataaatacacgcttagtctagcagatttttggtaaaatgcttagcctttgtacgattttatggctaattttaataacaagtgaataaaaactaccaactttttggtaaatttgattttaagtataagtggttcatgtaatttatatgccaaaaagtatgtgcataaaatcaatcaaatggtttatctgtcaatttgatgagtgggtattgagggtttttgcttcatgattaaaatcattgagaattaattactatcataattactataatattacagatatgtaaataaaaaaccattcatcatttacttttgtaattgcttaattttttttgagcgaataaaaggcggttttgtttatcaattgttgccagcgcttttaagttgccataaaatcagtcacaatagagttataaaacaagtggcttcaagcaacttgttgtttttcttaaggacggcatcggcattttgctgatggataatgaaatttaaatttaaaatgacctatggagtgacttatgagcttaattaataaattaaatgaacgcattacgccgcatgtcttaacttcgattaaaaatcaagatggcgataatgctgataaatctaatttgttaaccgcattttataccatttttgcaggacgcttgagtaatgaagatgtgtatcagcgtgccaatgctttgcctgataatgagcttgagcatgggcatcatctgctcaatgttgcttttagtgatgtttcaactggtgaagatcagattgcttctttgagtaatcaattagccgatgaatatcatgtttcgccagtaacggcacgcaccgcaatcgcaacggcagcacctttggctttggcacgcattaacattaaagagcaagcaggtgtattgtctgtaccgtcttttattcgtactcaattggctaaagaagaaaaccgtttgccaacttgggcgcatactttattgccagcagggctatttgcaaccgctgccacaaccaccgccgagcctgtaacgacagcctctgctgttgtgaaagagcctgtcaaaccaagtgttgtgacagaaccagttcatccagctgcggctaccaccccagtcaaaacaccaactgcccggcattacgaaaacaaagaaaaaagtccttttctaaaaacgattctaccgattattggattgattatttttgcaggcttggcatggcttttgttaagagcatgtcaagacaaaccaacacctgttgcggcacctgttgcgacagatacagcacctgtggtagcggataatgctgtacaggcagacccaacacaaacaggtgttgcccaagcacctgcaacgcttagcttgtctgttgatgaaacgggtcaagcgttgtactcgcaccgtgctcaggttggtagtgaagagcttgcaggtcatatccgtgcagctattgctcaagtctttggcgtacaagatttaaccattcaaaataccaatgtacataccgctacgatgccagcggcagaatacttaccagcaattttgggtttgatgaaaggtgtaccaaattcaagcgttgtgattcatgatcatacggtacgctttaatgcaaccacgccagaagatgtagcaaaactggtagagggtgctaaaaatattctacccgctgattttactgtagaagcagaacctgaacttgatattaatactgcggttgccgatagtattgaaacagcgcgtgttgctattgttgctttgggtgatacggttgaagaaaatgagatggatattttaatcaatgcattaaatacccaaatcattaactttgctttagactcaaccgaaattccccaagaaaataaagaaatcttggatttggctgccgaaaaattaaaggcagtgcctgaaacaactttgcgtatcattggtcatacagacactcaaggcacgcatgagtataatcaagatttatcagaatctcgtgctgctgctgttaaagagtatttggtatcaaaaggtgttgctgctgaacgtttgaacactcaaggtgcaagttttgattatccagttgcatcaaatgctaccgaacaaggtcgcttccaaaaccgtcgtattgagtttgtacttttccaagaaggtgaagcaattactcaagtcggtcatgctgaagatgcaccaacacctgttgcacaaaactgatcattttgttattggttatgagttttagattgggccaaatgaatgataatataccaatcttacaagtacttttaataaccaaaaccaaccgtaatcaacccaagaaccaaattacccatcggtcatttggttcttgggtagtttttattggctctcaatatatgatgtagaccaatttgacccaaaatagatcagagtttgggtcttggatttgcgaccatatcgtataactgacatatcttgaacacaaaaaagcataaaatga

SEQ ID NO: 41 - protein, 553 aa, Moraxella catarrhalis
MSLINKLNERITPHVLTSIKNQDGDNADKSNLLTAFYTIFAGRLSNEDVYQRANALPDNELEHGHHLLNVAFSDVSTGEDQIASLSNQLADEYHVSPVTARTAIATAAPLALARINIKEQAGVLSVPSFIRTQLAKEENRLPTWAHTLLPAGLFATAATTTAEPVTTASAVVKEPVKPSVVTEPVHPAAATTPVKTPTARHYENKEKSPFLKTILPIIGLIIFAGLAWLLLRACQDKPTPVAAPVATDTAPVVADNAVQADPTQTGVAQAPATLSLSVDETGQALYSHRAQVGSEELAGHIRAAIAQVFGVQDLTIQNTNVHTATMPAAEYLPAILGLMKGVPNSSVVIHDHTVRFNATTPEDVAKLVEGAKNILPADFTVEAEPELDINTAVADSIETARVAIVALGDTVEENEMDILINALNTQIINFALDSTEIPQENKEILDLAAEKLKAVPETTLRIIGHTDTQGTHEYNQDLSESRAAAVKEYLVSKGVAAERLNTQGASFDYPVASNATEQGRFQNRRIEFVLFQEGEAITQVGHAEDAPTPVAQN

SEQ ID NO: 42 - DNA, 519 bp, Moraxella catarrhalis
atgatgttacatattcaaattgccgccgctgccgccgctttatcggtactaacttttatgacaggctgtgccaataaatcaacaagtcaagttatggttgctcctaatgcacccacaggttacactggggttatctatactggtgttgcacctttggtagataatgatgagaccgttaaggctctggcaagcaagctacccagtttggtttattttgactttgattctgatgagattaaaccgcaagctgctgccatcttagacgaacaagcacaatttttaaccaccaatcaaacagctcgtgttttggttgcaggtcataccgatgagcgtggtagtcgtgagtataatatgtcactgggggaacgccgtgcggtggcggtacgcaactatttgcttggtaaaggcattaatcaagccagcgttgagattatcagttttggtgaagaacgccctatcgcatttggcacaaatgaagaagcatggtcacaaaatcgtcgtgctgaactgtcttattaa

SEQ ID NO: 43 - protein, 172 aa, Moraxella catarrhalis
MMLHIQIAAAAAALSVLTFMTGCANKSTSQVMVAPNAPTGYTGVIYTGVAPLVDNDETVKALASKLPSLVYFDFDSDEIKPQAAAILDEQAQFLTTNQTARVLVAGHTDERGSREYNMSLGERRAVAVRNYLLGKGINQASVEIISFGEERPIAFGTNEEAWSQNRRAELSY

SEQ ID NO: 44 - DNA, 675 bp, Moraxella catarrhalis
atgaaaattaaagcattgggtgttgtgctgttggcatcaagtatggctttggcaggttgtgcaaatacaggcacaactggcaatggcacaggatttggtggtgctaatgtcaataaggcggtgattggggctgtggcaggtgcacttggcggtactgccatttcaaaagcaactggtggcgaaaaaacaggtcgtgatgccattttgggggcggcagttggtgcagcagcaggggcgtatatggagcgtcaagcaaagcagattgagcaacaaatgcaaggaacgggcgtgactgtaacccacgataccgacacgggtaatattaatctaactatgccaggtaatattacttttgctcatgatgacgatactttaaacagtgcatttttgggtcgtttaaaccagctggctaatacgatgaatcagtatcatgaaacaacgattgtcattgtaggacatacagactcaacgggtcaagcggcttataatcaagagctgtctgagcgtcgagcggattcagtgcgttattacttgattaatcaaggcgttgatccatatcgtattcagacagtggggtatggtatgcgacaaccgattgcatcgaatgcaaccgaagcaggtcgtgctcaaaatcgccgtgttgagctgatgattttagcaccgcagggtatgtaa

SEQ ID NO: 45 - protein, 224 aa, Moraxella catarrhalis
MKIKALGVVLLASSMALAGCANTGTTGNGTGFGGANVNKAVIGAVAGALGGTAISKATGGEKTGRDAILGAAVGAAAGAYMERQAKQIEQQMQGTGVTVTHDTDTGNINLTMPGNITFAHDDDTLNSAFLGRLNQLANTMNQYHETTIVIVGHTDSTGQAAYNQELSERRADSVRYYLINQGVDPYRIQTVGYGMRQPIASNATEAGRAQNRRVELMILAPQGM

SEQ ID NO: 46 - DNA, 3650 bp, Haemophilus influenzae
gagttttttatttagttaagtatggagaccaagctggaaatttaacttgaccatcacttcctggaaggctcgccttaaagcgaccatctgcggaaaccaattgtagcacctttcctaagccctgtgtagaactataaataatcataattccatttggagagaggcttgggctttcgcctagaaaagatgtactaagtacctctgaaacgcccgttgtgagatcttgtttaactacattattgttaccattaatcatcacaagtgtttttccatctgcactaatttgtgcgctaccgcgaccacccactgctgttgcactaccaccgcttgcatccattcgataaacttgtggcgaaccacttctatcggatgtaaataaaattgaatttccgtctggcgaccacgctggttcagtattattacccgcaccactcgtcaattgagtaggtgtaccgccatttgctcccataacgtaaatattcagaacaccatcacgagaagaagcaaaagctaaacgagaaccatctggcgaaaaggctggtgcgccattatgcccttgaaaagatgccactactttacgtgcgccagaatttaaatcctgtacaacaagttgtgattttttattttcaaacgatacataagccaaacgctggccgtctggagaccaagctggagacataattggttgggcactacgattgacgataaattgattatagccatcataatctgctacacgaacttcataaggttgcgaaccgccatttttttgcacaacataagcgatacgagttctaaaggcaccacggatcgcagttaatttttcaaaaacttcatcgctcacagtatgcgcgccatagcgtaaccatttatttgttactgtatagctattttgcattaatacagtccctggcgtacctgatgcaccaaccgtatcaattaattgataagtaatactataaccattacccgatggaaccacttgcccaattacaattgcgtcaattccaatattcgaccaagcctcaggatttacctctgcagctgaagttgggcgttgaggcatttgagaaaccgcaataggattaaacttaccactgttacgtaaatcatctgcaacaattttactaatatcttctggtgcagaaccaacaaatggcacgacagcaataggacgcgcaccatcaaccccttcatcaatgacaatgcgtacttcatcgccagcgaatgcattgcttccaacagcaagtacaatcgcgaatacgctcactaaacgttttaataatttcattttgttacctttaaaatttaacaataaatttttctaaagaattatcgaatatcaaagtcaataattggtgatttatatttttcataaatttcatctgatggcgcagctggaacttttttcgttctagccaccgcacttaatgcagctgaacaaatatcatcagagcctgaaattttttgataccccaagattgtgccatctcgacctaattgaattttaatacgacaaacctttcctgcaaaatttggatcttttaagaaacgacgttgaatctctttcttaattacacctgcgtattgatccccaaccttaccaccatcgccagagccaagtgcagcaccgctaccttgagttccacctttatttgtgtttccccctttagatgcactaccgccaccaatatctccgccatttaagaaatcatctaggcttgcttgatctgctttacgtttcgcttccgtagcagctttagcttctgcatcagcttttgcttttgcctctgctgccgctttcgcttttgcctccgcttcagcctttgctttagcttcagcaacggcttttgccttagcttcggcttctagtttcgccttagcttccgcctcttgttttgctttttgagcagcaatttctgctgctttcgctttagcctcttcttcagcttgttttgccgcggcagctaaacgtttagcctctgcatctgcttttaattttgcagcttcagccgcttgtttagccttagcctcttcagcttgcttctgtttttccaacgcttcttgacgagcttgctcttgttgtttttttatttcttgctgacgttgctgttcttgctgacgttttaactcttcttgtcgttgaacttcttgttgatgcttaatctcttcttgattaggctcaggtggtttttcttccacaacaggttctgggcgtttttgtttatccgcttgcccttttttttgttgttgaatacgcccccattcctgagcagccgtaccagtatcaacaatcactgcccctattacatctccttcaccttctccaccacccataatttcaacagtgtgataaagtgagcttaaaatcaataagccaaacaagataaagtgcaaaaggatagaaatagcaaaagcattgattcctttcttttgtcgattattttgcacgtgttacctacttagctaaatgggatttgtcattaaacctacagatttaatgcctgcaagatgaagtaaattcaatgccttaatcacttcttcataaggtacttctttagctccgcctactaaaaatagcgtattattatccttatcaaattcctgtctagataattgagtaaccatttcttctgttaaaccttcttgacgttctccgccaatagaaatcgcatattttccaatgcctgccacttcaagaatgacgggtactttatcttcattagaaacctcttggctttgcacagaatcaggcaattcaacttgaacgctttgactaataataggggcggttgccataaaaattaacactaaaactaaaagcacatctaaaaaaggcacaatattaatttcagatttaattgctttacgctgacgacgagccatatattcctctaaaattttaacttatttttaccgcactttttcttcaaagtgcggtcaattttccctatattttagtgaggggctttaccaaaggcttgacggtgtaaaatcgtcgtaaattcatcaataaaattaccgtaatcttgttcaatggcattcactcgtaagcttaaacggttataagccattactgcaggaattgcggcaaataaaccaatcgcagtggcaatcaaggcctcagcgatacctggcgctaccatctgtaacgttgcttgttttgcaccacttaatgccataaaagcgtgcatgataccccaaacagtgccgaataaaccaatataagggctaacagatgccactgtggctaaaaatggaactcggttttccaaactttcaatctcacggttcatcgcaagattcatcgcgcgcattgtgcctttaataatcgcttcaggtgcatctggatttacttgttttaaacgtgaaaattctttaaatcccacgcaaaaaatttgttcgctgcccgttaatccatcgcgacgattagatagcccttcataaagtttatttaaatcttctcctgaccagaaacgatcttcaaacgtacgcgcttcttttaaggcattcgttaaaatacgactacgttgaatgataattgcccaagatatgattgagaaagaaatcaaaatcacaattaccagttgcacaacaatacttgcttttagaaaaagatctaaaaaattcaattctgcagtcattgcata

SEQ ID NO: 47 - DNA, 4600 bp, Moraxella catarrhalis
gggtgatagcgcacctcaacaggatagctacgaccctcgacaatatacacaggtgcaggtttgccattcgctgcaaaatagtcagaaaacctttgggtgtctaaagtggcggaggtgatgataacttttagatcagggcgtttgggtaaaagacgctttaaatagcccatgataaaatcaatatttaagctacgctcatgtgcttcatcaatgatgatggtatcataatttgccaaaaacttatcagagcccaattcagcaagtaaaatcccatctgtcatcagcttgacaatagagtgcttgccaccttcttcggtgaagcgaatcttaaaactcaccgtctgaccaagtggctcgccaagctcttcagcgatacgcatcgctaccgagcgtgcagccaatcggcgtggctgtgtgtggccaatttgacctgtgatgccacgccctgccatcatagcaagcttaggcagttgcgtggttttgccagaacccgtctcacctgcgataatcaccacttgatgatcacggatcgcttgaattagcgtatcggcttcagcagtcacgggcaaatcatgattaagtttttctgatagattttttggtatgctatccatacgattggcgacctgttcggcagatcgctcatagatagcatcatagcgtattttgcacttagtttttagatcgcctgtggtagaattcattttctgttttagtttatttaaataatgtctgtctttggcaaggactggtaaattatcggtagaatgcatatttttaaatgatagttatcttataaagggtatgaaaaagcatcaatttaagtacattgatacatcagattttattttattcatgggtctatatgagggcttggacgcatgaataaaccatgtattgtaaataaaatcatcaaaacctgcaattttctatttaaatggcgattttagggcgatagacaagcgatgactttttgcccatctgtcgcaaatttattaacttatgctataatgccaagtatctttttttgcctattgtgattgtcaattatgaacgaatccattagcctaatctcgctggtcattgaagcaagcgttgttgttaaattggtcatggcgatactgcttttgctgtctacaatcagttgggtactgatttttcatctgggtaccaaaattggcggtattgccaagtttgataagcgatttgagcgatggttttggactgatgatatcgatcatcagctgtctgttgtgcaagcagaatcagagcgtgcagggcttgagctgattttttatacaggtttttatgatcaaaatcaccaagaccaagattcttcactaagtgatgataaaaaagtgcaaatcgttgagcgtcgcttgcgtatggcattaggcagtgagcaggtgcatcttgaaaaaggattatcaacgcttgcaacgattggttctgtttcaccttatatcggactatttggtacagtatggggcattatgaatgcatttattggcttgggtcaagccgaatcggttggtcttgcaaccgttgcaccgagcattgctgaggcattgattgcaacagcacttggtttatttgcggccattcctgcgacgatggcatataatcactttgccaccaaatccaatacactgtatgaaaatcgtagcctattttgtgaaggcttaataagtgcattggtgacaaatctggcaaaaaagaacaccgcatcaactttatagagcatactattttatagagcatattatggtaacttccaatcgattcgctcgtcgccaaagaccgctaaatagtgacatgaatgttgtgccttacattgatgtgatgttggtgcttttggtgatatttatcgtaacagcaccaatgcttgctacaggtattgaggtatcactgccaaaagagcagaccaaacccatcacacaagctgacaagctgcctgtcattgtcagcattcaggcagatggcaatctgtatgtcagccataaaaatgccatcgatgtgccaatcacgcctgacaagctagataccctgctacgccagatgcaccaagacaataccgatttacaagtgatggtcaatgccgatgcagataatgcctacagccgaattatgcagattatggcattgattcaaaatgttggtatcacccaagtgagtttgcttagcgaatctgttcaataatgcatgataattcataaggcaaatcaatcgatgcgtttatccgataatcatccaacagtcaattttgataaatctgcgctaattttaccaattttagccagtgttttattacataccgtcatcatcatagcggtagcagcaccactgattacaccgcctactaagcctaatactactattcagaccgctttggtaggtcaagaggcttttaatcgtgccaagacggccttgagcaatcatcatgccaatcaaaacaagccaactgccaccaacacttcaagtaccatcactgccaatgataatgataatgcatttatgcaagctcaaaatcagcatcgttatcacccacaggtttctacttctgccaccacgacccaagcgtatcatccaccacccaactcagcaccctttgaatcaaattcaccaaatatacaaaatcaaccaacaaacgctcacgccaagctggctgaatattctaatcatgtctcagaccttgagcagtcaaatcataccgagtctacgccaagccgagcacaaatcaatgccgccatcacctcggtcaaacatcgtattgaagccatttggcaacgctatcctaagcagcccaatcaaaccatcacctttcaggttaatatgaatcaacaaggcgatgtgacctcaatccaattcggtggtggccatcctgatttgcgtgaatctgtagaagcggcggtatatgctgccgcaccattttatgaacttggcggtatgcgtgacagtatccgcctgcagttcaccacagagcagctaattatggataataaccaaacaaccaatgagcctaatcactaatcgccatggagtttttatgaaatcacccattaccaaagtttgccttgctctgaccataagcttttctgccgctttgacgcacacttatgctgatgatgaattgattgtgattagcgaacaagttgctccgagtcaataccccgtggcagtcatgcctttttcagaagctcatcaaatgagtcattatctaagcctggcaggtcttggtactactcaccaaaacctgccacagcacactcagacgaatagcgacattctgaataatctgaccgcatggcgtaaccgaggatttgaatatattattttggcacagtcgcatcaaattttgggaaataagcttgcaattaactatgaaattattgatactgccaatggtttggtaagcgtcaagcatacccaaattagcgataaccaccctgcttctatccaagctgcctatcgtcaaatcagcgatacaatctatcaaatcatcacaggccagccatcagatttgatgggtaaaatcgcctatgtggaagaaagcggatcgccacaaaataaaatctcatctcttaaattgattgatccaagcggtcagcttatccgtacgctagataccgtcaatggatcaattataacgccgacattttcccccgatggcttgagtattgcttatagtgtacaaacaaaaaataatctgcccatcatttatattgtgtctgtatcaggtggcacaccaaagctcgtcacgccattttggggtcataatttggcaccaagtttttcaccagatggtagcagtatcttattttcaggtagccacgagaataataacccgaacatttatcgtcttaatttacataccaatcacttagatacgctcactacattcaacggtgctgagaatgcaccaaattatttggcagatgcgtcaggatttatttatactgctgataaaggtacacgccgccaaagcctatatcgctatgattttggcacgacgcatagcacccaaatcgcctcttatgccaccaatccacgcttaagcccagatggatcaaagcttgtatatttatcaggtggacaaatcatcatcgccaataccaaaggccgtatccaacaaagttttagggtgttaggcactgatgtatcagccagcttttcaccatcaggcacacggattatatatacatccaaccaaggcaataaaaaccagctgatgatccgttcgctatcaagtaatgccatacgcaccatcccaacatcaggcacggtgcgtgatccgatttggtcaaaataatgccaatgagtatcccaactaaggcgacagtcggctatacccaaaggcggttatttatggtcagtatgacagttggcctgatcagcttgagtgggtgtcagcacattcaagtgaccaaaagcccaataccgatcatcatccatagccatacaaaatcgccatctcagcctaaacctacaccaactgacgccgtgcctaccaaaaaccgcccaatctccccaccaacacaaaagtccaatacgatatttattttggaagattggttttaggcagttttggtagattcaaaat

SEQ ID NO: 48 - DNA, 32 bp, Artificial Sequence
  primer
gcccacaagcttatgaccaaacagctgaaatt

SEQ ID NO: 49 - DNA, 29 bp, Artificial Sequence
  primer
ccggaattcttagtgttggtgatgattgt

SEQ ID NO: 50 - DNA, 29 bp, Artificial Sequence
  primer
ggcggatccttagaacagggttttggcag

SEQ ID NO: 51 - DNA, 29 bp, Artificial Sequence
  primer
cggggatcccaagacaacctgaaagtatt

SEQ ID NO: 52 - DNA, 38 bp, Artificial Sequence
  primer
cgcggatccgccgtctgaaacctgtgacggaagatcac

SEQ ID NO: 53 - DNA, 37 bp, Artificial Sequence
  primer
cgcggatccttcagacggcccaggcgtttaagggcac

SEQ ID NO: 54 - DNA, 21 bp, Artificial Sequence
  primer
catgatagactatcaggaaac

SEQ ID NO: 55 - DNA, 20 bp, Artificial Sequence
  primer
cagtacctggtacaaaatcc

SEQ ID NO: 56 - DNA, 38 bp, Artificial Sequence
  primer
gctctagagcttcagcagtcacgggcaaatcatgatta

SEQ ID NO: 57 - DNA, 38 bp, Artificial Sequence
  primer
cggagctctgctcaaggtctgagacatgattagaatat

SEQ ID NO: 58 - DNA, 37 bp, Artificial Sequence
  primer
cgggatcccagcgagattaggctaatggattcgttca

SEQ ID NO: 59 - DNA, 38 bp, Artificial Sequence
  primer
cgggatccaatgttggtatcacccaagtgagtttgctt

SEQ ID NO: 60 - DNA, 21 bp, Artificial Sequence
  primer
atcggcgtggctgtgtgtggc

SEQ ID NO: 61 - DNA, 21 bp, Artificial Sequence
  primer
accgaattggattgaggtcac

SEQ ID NO: 62 - DNA, 21 bp, Artificial Sequence
  primer
gcgattcaggcctggtatgag

SEQ ID NO: 63 - DNA, 21 bp, Artificial Sequence
  primer
ttgtgcaatgtaacatcagag

SEQ ID NO: 64 - DNA, 38 bp, Artificial Sequence
  primer
cctctagacgcttattataacataaatcagtctaactg

SEQ ID NO: 65 - DNA, 38 bp, Artificial Sequence
  primer
aaggtaccagcagaagtagccaatgggcaaaacattgc

SEQ ID NO: 66 - DNA, 38 bp, Artificial Sequence
  primer
ccggatccttaacggtattgtggtttgatgattgattt

SEQ ID NO: 67 - DNA, 38 bp, Artificial Sequence
  primer
aaggatccgcgcaaatgcgtgaattcccaaatgcaact

SEQ ID NO: 68 - DNA, 50 bp, Artificial Sequence
  primer
ccggaattcaaagtgcggtagatttagtcgtagtaattgatttacttatg

SEQ ID NO: 69 - DNA, 28 bp, Artificial Sequence
  primer
ctagtctagaacgttgctgttcttgctg

SEQ ID NO: 70 - DNA, 27 bp, Artificial Sequence
  primer
cgcggatcccgcttcaggtgcatctgg

SEQ ID NO: 71 - DNA, 28 bp, Artificial Sequence
  primer
cgcggatccagacaggaatttgataagg

SEQ ID NO: 72 - DNA, 23 bp, Artificial Sequence
  primer
ccttactagaggaacaacaactc

SEQ ID NO: 73 - DNA, 20 bp, Artificial Sequence
  primer
gcctcttcagcttgcttctg

SEQ ID NO: 74 - DNA, 50 bp, Artificial Sequence; identical to SEQ ID NO: 68
  primer

SEQ ID NO: 75 - DNA, 28 bp, Artificial Sequence
  primer
ctagtctagaacgttgctgttcttgctg

SEQ ID NO: 76 - DNA, 45 bp, Artificial Sequence
  primer
ccggaattcaaagtgcggtagatttagtcgtaattcgctgaggcc

SEQ ID NO: 77 - DNA, 35 bp, Artificial Sequence
  primer
ctagtctagattatcgaatatcaaagtcaataatg

SEQ ID NO: 78 - DNA, 31 bp, Artificial Sequence
  primer
cgcggatccttcttctgtttaaaccttcttg

SEQ ID NO: 79 - DNA, 27 bp, Artificial Sequence
  primer
cgcggatccaagcaaaggctgaagcgg

SEQ ID NO: 80 - DNA, 18 bp, Artificial Sequence
  primer
cgctgaggccttgattgc

SEQ ID NO: 81 - DNA, 22 bp, Artificial Sequence
  primer
gtacaatcgcgaatacgctcac

SEQ ID NO: 82 - DNA, 45 bp, Artificial Sequence; identical to SEQ ID NO: 76
  primer

SEQ ID NO: 83 - DNA, 35 bp, Artificial Sequence; identical to SEQ ID NO: 77
  primer

SEQ ID NO: 84 - DNA, 47 bp, Artificial Sequence
  primer
gatgaattcaaagtgcggtagatttagtcgtagtaattaataactta

SEQ ID NO: 85 - DNA, 31 bp, Artificial Sequence
  primer
ctagtctagaaggtttccataatgtttccta

SEQ ID NO: 86 - DNA, 35 bp, Artificial Sequence
  primer
cgcggatccctaaaaagttacatcagaatttaagc

SEQ ID NO: 87 - DNA, 29 bp, Artificial Sequence
  primer
cgcggatccgcatttggtaaagcaaactt

SEQ ID NO: 88 - DNA, 47 bp, Artificial Sequence; identical to SEQ ID NO: 84
  primer

SEQ ID NO: 89 - DNA, 31 bp, Artificial Sequence; identical to SEQ ID NO: 85
  primer

SEQ ID NO: 90 - protein, 346 aa, E. coli
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 91 - protein, 240 aa, Neisseria meningitidis
MTKQLKLSALFVALLASGTAVAGEASVQGYTVSGQSNEIVRNNYGECWKNAYFDKASQGRVECGDAVAAPEPEPEPEPAPVVVVEQAPQYVDETISLSAKTLFGFDKDSLRAEAQDNLKVLAQRLGQTNIQSVRVEGHTDFMGSDKYNQALSERRAYVVANNLVSNGVPVSRISAVGLGESQAQMTQVCEAEVAKLGAKVSKAKKREALIACIEPDRRVDVKIRSIVTRQVVPAHNHHQH

SEQ ID NO: 92 - protein, 236 aa, Neisseria gonorrhoeae
MTKQLKLSALFVALLASGTAVAGEASVQGYTVSGQSNEIVRNNYGECWKNAYFDKASQGRVECGDAVAVPEPEPAPVAVVEQAPQYVDETISLSAKTLFGFDKDSLRAEAQDNLKVLAQRLSRTNVQSVRVEGHTDFMGSEKYNQALSERRAYVVANNLVSNGVPASRISAVGLGESQAQMTQVCQAEVAKLGAKASKAKKREALIACIEPDRRVDVKIRSIVTRQVVPARNHHQH

SEQ ID NO: 93 - protein, 173 aa, E.coli
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 94 - protein, 78 aa, E.coli
MKATKLVLGAVILGSTLLAGCSSNAKIDQLSSDVQTLNAKVDQLSNDVNAMRSDVQAAKDDAARANQRLDNMATKYRK

SEQ ID NO: 95 - protein, 240 aa, Neisseria meningitidis; identical to SEQ ID NO: 91

SEQ ID NO: 96 - protein, 236 aa, Neisseria gonorrhoeae; identical to SEQ ID NO: 92

SEQ ID NO: 97 - protein, 346 aa, E.coli; identical to SEQ ID NO: 90

SEQ ID NO: 98 - protein, 173 aa, E.coli; identical to SEQ ID NO: 93